(19) United States
(12) Patent Application Publication
(10) Pub. No.: US 2026/0102478 A1
(43) Pub. Date: Apr. 16, 2026

(54) Vaccines and Antibodies for the Treatment and Prevention of Neurodegenerative Disorders and Inflammation Related Health Conditions

(71) Applicant: Longhorn Vaccines and Diagnostics, LLC, Bethesda, MD (US)

(72) Inventors: Jeffrey D. Fischer, Washington, DC (US); Clara J. Sei, Germantown, MD (US); Gerald W. Fischer, Bethesda, MD (US)

(73) Assignee: Longhorn Vaccines and Diagnostics, LLC, Bethesda, MD (US)

(21) Appl. No.: 19/357,162

(22) Filed: Oct. 14, 2025

Related U.S. Application Data
(60) Provisional application No. 63/706,770, filed on Oct. 14, 2024.
(60) Provisional application No. 63/709,551, filed on Oct. 21, 2024.
(60) Provisional application No. 63/738,652, filed on Dec. 24, 2024.

(51) Int. Cl. A61K 39/085; A61K 39/00; A61K 39/04; A61K 39/07; A61K 39/09; A61K 39/385; A61P 31/04; A61P 31/16; C07K 16/12; G01N 33/68

(52) U.S. Cl. CPC A61K 39/085; A61K 39/04; A61K 39/07; A61K 39/092; A61K 39/385; A61P 31/04; A61P 31/16; C07K 16/1271; C07K 16/1275; C07K 16/1278; C07K 16/1289; G01N 33/6893; A61K 2039/54; A61K 2039/572; A61K 2039/6037; A61K 2039/6075; A61K 2039/6087; A61K 2039/70; C07K 2317/24; C07K 2317/31; C07K 2317/94; G01N 2333/4737; G01N 2333/5753; G01N 2800/52

(57) ABSTRACT

The invention is directed to immunological compositions of one or more peptides containing epitopes of PGN, LTA and LPS molecules that induce an immunological response in a mammal, and to multiple antibodies that bind to these epitopes. Immunological compositions and antibodies disclosed herein can be used in the treatment and/or prevention of human health disorders such as bacterial sepsis, inflammation, cancers, tumors, inflammatory diseases and disorders, and neurodegenerative disorders such as, but not limited to Alzheimer's disease, frontotemporal dementia, chronic traumatic encephalopathy (CTE), Lewy body dementia and/or limbic predominant age-related TDP-43 encephalopathy (LATE).

### REFERENCE TO RELATED APPLICATIONS

This application claims priority to U.S. Provisional Application No. 63/738,652 filed Dec. 24, 2024, U.S. Provisional Application No. 63/709,551 filed Oct. 21, 2024, and U.S. Provisional Application No. 63/706,770 filed Oct. 14, 2024, the entirety of each of which is incorporated by reference.

### SEQUENCE LISTING

The instant application contains a Sequence Listing which is hereby incorporated by reference in its entirety. Said XML copy, created on Oct. 7, 2025, is named 3022_062_US_SL.xml and is 181,231 bytes in size.

### BACKGROUND

### 1. Field of the Invention

The present invention is directed to antigens composed of a plurality of epitopes including epitopes of PGN, LTA and LPS molecules or antibodies that are reactive to epitopes those molecules, that induce an immunological response in a mammal. Immunological compositions and antibodies disclosed can be used in the treatment and/or prevention of infections, sepsis, and inflammation related health conditions such as neurodegenerative disorders.

### 2. Description of the Background

A neurodegenerative disease is a disorder involving the progressive loss of neurons, typically of the brain. Such disorders include at least amyotrophic lateral sclerosis, multiple sclerosis, Parkinson's disease, Alzheimer's disease, Huntington's disease, multiple system atrophy, tauopathies, and prion diseases, with Alzheimer's disease being the most well-known.

Alzheimer's disease (AD) is a neurodegenerative disorder that is the cause of 60-70% of cases of dementia in the developed world. The disorder was first identified in 1906 by a psychiatrist named Alois Alzheimer. The symptoms of the disorder include problems with language, disorientation, mood swings, loss of motivation, self-neglect, and behavioral issues. Eventually even body functions are lost leading to death. Although the speed of progression can vary, the average life expectancy following diagnosis is three to twelve years.

The cause of Alzheimer's disease is unknown, but there is evidence of a genetic connection. In particular, there is often an accumulation of apolipoproteinin brain tissue, although this is usually determined after death. An addition theory is that there is a misfolding of certain proteins in the brain which is associated with the formation of amyloid plaques, neurofibrillary tangles, and loss of neuronal connections in the brain. Again, a definitive examination requires an analysis of brain tissue which can only take place after death.

No treatments can stop or reverse its progression, though some may temporarily improve symptoms or slow the progression of the disease. Those affected are reliant on family members, caregivers, and/or health care workers for assistance. Behavioral psychosis due to dementia are treated with antipsychotics.

As of 2020, there were approximately 50 million people worldwide with Alzheimer's disease. Most are over 65 years of age, although up to 10% of cases are early-onset impacting those in their 30s to mid-60s. Alzheimer's financial burden on society is large, with an estimated global annual cost of US$1 trillion. It is ranked as the seventh leading cause of death worldwide.

Inflammation is a biological response to stimuli including many pathogens, bodily damaged to cells, and other materials. Signs of inflammation include heat, pain, redness, swelling, and loss of sensory function. Inflammation is a protective response involving immune cells, blood vessels, and molecular mediators, all considered a mechanism of innate immunity. The function of inflammation is to eliminate the initial cause of cell injury, clear out damaged cells and tissues, and initiate tissue repair. Too little inflammation could lead to progressive tissue destruction by the harmful stimulus (e.g. bacteria) and compromise the survival of the organism. However, inflammation can also have negative effects. Too much inflammation, in the form of chronic inflammation, is associated with various diseases, such as hay fever, periodontal disease, atherosclerosis, sepsis, and osteoarthritis.

Acute inflammation involves the initial response of the body to harmful toxins that induce shock and organ toxicity and if unchecked proceeds to death. This may mature into an inflammatory response, involving the local vascular system, the immune system, and various cells in the injured tissue. Chronic inflammation is prolonged inflammation and leads to a progressive shift in the type of cells present at the site of inflammation, such as mononuclear cells, and involves simultaneous destruction and healing of the tissue, and can be classified as Type 1 and Type 2 based on the type of cytokines and helper T cells (Th1 and Th2) involved. Toxin induced sepsis and shock may be silent and set-up brain and other organs for chronic inflammation. Bacterial toxin induced illness may also be silent and set-up brain and other organs for chronic inflammation. It is now understood that viruses (such as influenza and SARS-CoV-2) as well as bacteria and bacterial toxins can induce inflammation, including neuroinflammation.

Inflammatory abnormalities include a group of disorders that underlie a vast variety of human diseases. The immune system is often involved with inflammatory disorders, as demonstrated in both allergic reactions and some myopathies, with many immune system disorders resulting in abnormal inflammation and sepsis, a life-threatening condition that arises when the body's response to infection results in damage, often irreversible, to the patient's own tissues and organs. Non-immune diseases with causal origins in inflammatory processes include cancer, atherosclerosis, and ischemic heart disease. At present, anti-inflammatory medicines can improves symptoms, but a more rapid and positive treatment is mostly unavailable.

Inflammation can be classified as either acute or chronic. Acute inflammation involves an initial response of the body to harmful stimuli, such as an infection. Inflammation manifests itself by the movement of plasma, including leukocytes and granulocytes, from the circulatory system to injured tissues. A series of biochemical events propagates and matures the inflammatory response, involving the local vascular system, the immune system, and various cells in the injured tissue. Chronic inflammation involves a prolonged inflammatory response leading to the involvement of more mononuclear cells, which can cause the destruction and/or healing of the damaged tissue. Alternatively, chronic inflammation can be the disorder itself when no damaged or infected tissue remains, but the immune response continues unabated. This form of inflammation is common in diabetics and obese individuals.

Chronic or systemic inflammation is the result of a prolonged inflammatory response. This prolonged response involves the release of pro-inflammatory cytokines from immune-related cells and the chronic activation of the innate immune system. Chronic inflammation contributes to the development or progression of cardiovascular disease, cancer, diabetes mellitus, chronic kidney disease, non-alcoholic fatty liver disease, autoimmune and neurodegenerative disorders, and coronary heart disease.

Compositions and method for the treatment of Alzheimer's, other neurodegenerative disorders, inflammation including chronic inflammation, sepsis, and other related diseases and conditions are greatly needed.

Cancer as a term refers to abnormal cell growth which may invade and spread to other parts of the body. A corollary to cancer is a benign tumors, which also comprises abnormal cell growth that does not spread beyond a particular organ or area of the body There are hundreds of varieties of cancers and tumors, often named for the cell type of the origin of the proliferation.

Common forms of cancer in males are lung cancer, bladder cancer, prostate cancer, colorectal cancer, and stomach cancer; and in females, colorectal cancer, ovarian, lung cancer, and cervical cancer. Common to both includes melanoma and bladder cancer. Other forms of cancer include acute lymphoblastic leukemia, brain tumors, non-Hodgkin lymphoma, The risk of cancer increases as more people live to an older age. Although a number of treatments are available for each form of cancer, including radiation and chemotherapies, more effective treatments are needed.

### SUMMARY OF THE INVENTION

The present invention provides new and useful compositions, as well as tools and methods directed to immunogenic compositions, vaccines and antibodies against one or more pathogens for treating and/or preventing infections in mammals such as humans, treating and/or preventing a viral, bacterial, fungal, or parasitic infection, treating various forms of cancer including pancreatic, breast, ovarian, bladder, non-small cell and lung cancers, treating various forms of inflammation including both acute and chronic inflammatory diseases and disorders, and in enhancing the immune system of a patient or mammal. In addition, the compositions, vaccines, and antibodies of this disclosure treat and prevent neurological disease, sepsis, inflammation including chronic inflammation and related disorders, and other related conditions.

One embodiment of the invention is directed to compositions comprising one or more peptides, preferably produced recombinantly or otherwise synthetically, containing epitopes and/or mimotopes of two or more different pathogenic organisms. Preferably the epitopes are of the molecules peptidoglycan (PGN), lipoteichoic acid (LTA), and lipopolysaccharide (LPS). Preferably compositions include three or more epitopes, one each from PGN, LTA, and LPS. Preferably the PGN molecule is obtained or derived from a gram-positive microorganism of a spp. of the genera Mycobacteria, Staphylococcus, Bacillus, or Streptococcus, the molecule LTA is obtained or derived from a gram-positive microorganism of a spp. of the genera Mycobacteria (traditionally an acid-fast microorganism), Staphylococcus, Bacillus, or Streptococcus, and the LPS molecule is obtained or derived from a gram-negative microorganism, which is of an enteric microorganism. Compositions may further contain one or more epitopes of a heat shock protein (HSP), one or more epitopes of a molecule of lipoarabinomannan (LAM), and/or one or more a T cell stimulating epitopes. Preferably the T cell stimulating epitope is obtained or derived from tetanus toxin, tetanus toxin heavy chain proteins, diphtheria toxoid, CRM, recombinant CRM, tetanus toxoid, Pseudomonas exoprotein A, Pseudomonas aeruginosa toxoid, Bordetella pertussis toxoid, Clostridium perfringens toxoid, Escherichia coli heat-labile toxin B subunit, Neisseria meningitidis outer membrane complex, Hemophilus influenzae protein D, Flagellin Fli C, Horseshoe crab Haemocyanin, and/or a fragment, derivative, or modification thereof. Composition may further contain adjuvants such as, for example, a toll-like receptor agonist (TLR), Freund's adjuvant, ALFQ, ALFQA, ALFA, AS01, AS01b, a liposome adjuvant, saponin, lipid A, squalene, and/or modifications, emulsions, nano-emulsions, derivatives, and combinations thereof. Contiguous peptides containing these epitopes include the sequence of one or more of any of the SEQ ID NOs. Preferably these compositions treat and/or prevent neurodegeneration disorders of mammalian brain tissue. Preferably the compositions disclosed here are vaccines or collections of one or more different monoclonal antibodies.

Another and related embodiment of the invention is directed to methods for the prevention and/or treatment of a neurodegeneration disorder and/or to reduce the inflammatory response, that may be used alone or together with conventional treatments options for these disorders. Method involved administering the composition disclosed herein to a subject, which is preferably a human male or female. The neurodegenerative disorder may be Alzheimer's disease, frontotemporal dementia, chronic traumatic encephalopathy (CTE), Lewy body dementia, limbic predominant age-related TDP-43 encephalopathy (LATE), or another neurodegenerative disorder. Preferably, administration of the compositions disclosed herein prevents the accumulation of amyloid-beta particles (Aβ) in mammalian brain tissue. Preferably, administration is oral, sub-cutaneous, intra-muscular, intradermal, or intra-nasal.

Another and related embodiment of the invention is directed to methods for the treatment of various forms of cancer including pancreatic, breast, and non-small cell and lung cancers with the compositions disclosed here. Methods involve the administration of immunogenic compositions such as collections of peptides containing epitopes of one or more of the molecules of peptidoglycan (PGN), lipoteichoic acid (LTA), and lipopolysaccharide (LPS), and/or collections of antibodies that are specifically reactive to the one or more of these epitopes. Preferably, the immune system provide an opsonophagocytic response. Preferably the composition and/or to reduce the proliferation of cancer cells, and may be used alone or together with conventional treatments options for these disorders. Method involved administering the composition disclosed herein to a subject, which is preferably an adult, a human male, a human female, a child or an infant. The types of cancer which can be treated include, but are not limited to, lung cancer, bladder cancer, ovarian cancer, pancreatic cancer, prostate cancer, colorectal cancer, stomach cancer, breast cancer, colorectal cancer, cervical cancer, melanoma, and related diseases and disorders. Preferably, administration of a composition as disclosed herein prevents the cascade of proliferation inducing agents and shuts down development of cancer. Preferably, administration is oral, sub-cutaneous, intravascular, intra-muscular, intradermal, or intra-nasal.

Another and related embodiment of the invention comprises a collection of antibodies that are specifically reactive to the one or more peptides containing epitopes of various molecules attributed to pathogenic microorganisms or to molecules that are overly present. Such molecules are preferably peptidoglycan (PGN), lipoteichoic acid (LTA), lipopolysaccharide (LPS), and/or molecules such as cytokines. Preferably, the antibodies provide an opsonophagocytic response. Antibodies may be IgG, IgA, IgD, IgE, IgM, or fragments or combinations thereof, and may be polyclonal, bifunctional, monoclonal, and/or humanized.

Another and related embodiment of the invention comprises methods for prevention and/or treatment of a neurodegeneration, to reduce the inflammatory response including chronic inflammation, or to treat sepsis comprising administering the compositions and/or antibodies disclosed herein to a subject, such as, for example, a human male or human female. Preferably administration is oral, sub-cutaneous, intra-muscular, intradermal, or intra-nasal. Also preferably the neurodegeneration is of mammalian brain tissue such as occurs in Alzheimer's disease, frontotemporal dementia, chronic traumatic encephalopathy (CTE), Lewy body dementia and/or limbic predominant age-related TDP-43 encephalopathy (LATE). Preferably, these methods prevent the accumulation of amyloid-beta particles (Aβ) in mammalian brain tissue.

Another and related embodiment of the invention comprises one or more hybridomas that expresses the antibodies disclosed herein.

Another and related embodiment of the invention comprises methods for the prevention and/or treatment of a neurodegeneration of mammalian brain tissue in a subject comprising: administering a peptide sequence containing a combination of epitopes and/or mimotopes from a plurality of the molecules selected from the group consisting of peptidoglycan (PGN), lipoteichoic acid (LTA), and lipopolysaccharide (LPS), or a collection of antibodies that bind to the peptide; and monitoring procalcitonin in the blood of the subject, wherein a serum concentration of at least 0.5 ng/ml is indicative of neurodegeneration. Procalcitonin serves as a biomarker that determines a severity of systemic inflammation or bacterial infection in the subject. Administration of the peptide or the collection of antibodies can be continued or adjusted based on the level of procalcitonin in the subject. Preferably, procalcitonin levels are monitored post-administration to determine a therapeutic efficacy of the peptide or collection of antibodies. Further, C-reactive protein (CRP) levels in the blood of the subject may be monitored. Procalcitonin is preferably used to differentiate between bacterial and viral etiologies to determine appropriateness of therapy with anti-bacterial toxin immunological compositions.

Other embodiments and advantages of the invention are set forth in part in the description, which follows, and in part, may be obvious from this description, or may be learned from the practice of the invention.

### DESCRIPTION OF THE FIGURES

FIG. 1 The adjuvanted peptide vaccine that include bacterial toxins induce serum antibodies that bind well to the immunogen (peptide formulation).

FIG. 2A Peptides that include bacterial toxin epitopes induce serum antibodies that bind to gram-positive and gram-negative toxins (LPS, PGN, LTA) and to the whole bacteria. FIG. 2A discloses SEQ ID NO 124.

FIG. 2B Peptides that include bacterial toxin epitopes induce serum antibodies that bind to gram-positive and gram-negative toxins (LPS, PGN, LTA) and to the whole bacteria. FIG. 2B discloses SEQ ID NO 175.

FIG. 2C Peptides that include bacterial toxin epitopes induce serum antibodies that bind to gram-positive and gram-negative toxins (LPS, PGN, LTA) and to the whole bacteria. FIG. 2C discloses SEQ ID NO 130.

FIG. 3A Peptides that include bacterial toxins induce serum antibodies that show recognition of both gram-positive and gram-negative bacteria, including live mycobacteria.

FIG. 3B Peptides that include bacterial toxins induce serum antibodies that show recognition of both gram-positive and gram-negative bacteria, including fixed mycobacteria.

FIG. 4 Peptide vaccines that include bacterial toxins generate opsonic antibodies

FIG. 5 Binding activity of MAB JG7 to gram-positive bacteria.

FIG. 6 Binding activity of MAB JG7 to ultra-pure PGN (derived from E. coli) and whole E. coli.

FIG. 7 Binding activity of MABs JG7 and MD11 to various strains of gram-positive bacteria.

FIG. 8 Binding activity of MAB MD11 to ultra-pure PGN (derived from E. coli) and whole E. coli.

FIG. 9 Binding activity of MAB MD11 to various strains of gram-negative bacteria (E. coli).

FIG. 10 Opsonophagocytic activity of MAB MD11 against E. coli using macrophage cell line U-937s.

FIG. 11A Binding activity of MAB MD11 to live Mycobacterium tuberculosis (MTB) at mid-logarithm and stationary phase.

FIG. 11B Binding activity of MAB MD11 to ethanol-killed Mycobacterium tuberculosis (MTB).

FIG. 12A Binding activity of mAb DRG-5 BD11 to MTB HSP16.3 (TB Pep01) and ultra-pure PGN derived from S. aureus and E. coli-mAb titrated at 1:2

FIG. 12B Binding activity of mAb DRG-5 BD11 to MTB HSP16.3 (TB Pep01) and ultra-pure PGN derived from S. aureus and E. coli-mAb titrated at 1:3

FIG. 13A Binding activity of mAb DRG-5 BD11 to various live gram-positive and gram-negative bacteria at mid-Log phase of growth-line graph

FIG. 13B Binding activity of mAb DRG-5 BD11 to various live gram-positive and gram-negative bacteria at mid-Log phase of growth-bar graph

FIG. 14A Binding activity of mAb DRG-5 BD11 to various live E. coli strains at mid-Log phase of growth.

FIG. 14B Binding activity of mAb DRG-5 BD11 to various live E. coli strains at stationary phase of growth.

### DESCRIPTION OF THE INVENTION

Vaccinations and vaccines are often the best mechanism for avoiding an infection and preventing the spread of debilitating and dangerous pathogens, and also for the treatment and/or prevention of cancers and benign tumors. With respect to viral infections, parasitic infections and many bacterial infections, vaccinations may be the only effective option as preventative or treatment options are few and those that are available provide only limited effectiveness. Conventional vaccinations require a priori understanding or general identification of the existing antigenic regions of the pathogen. The pathogen itself is propagated and a suitable vaccine developed from heat-killed or otherwise attenuated microorganisms. With respect to cancers and tumors, treatment typically involve exposure of the proliferating cell to radiation and/or the administration of some form of chemotherapy.

It has been surprisingly discovered that antigens and/or collections of different antigens have been identified that are causative agents of a number of diseases and disorders. Pathogenic organisms located in the gut microbiome produce toxins that exacerbate and/or create health issues such as asthma, arthritis, diabetes, heart disease, stroke, cancers and neurodegenerative disorders. These organisms can also exacerbate the risk factors for these diseases which include high blood pressure, high cholesterol, elevated blood sugar levels. Specifically, the administration as compositions or collections of antibodies as disclosed herein generate or create a protective immune response against a particular viral or bacterial infection by eliminating or reducing the amount of pathogens in the GM. Thus, compositions as disclosed herein can be useful to treat and/or prevent a variety of cancers, degenerative neurological diseases. inflammation, and their related disorders and diseases.

The need for a vaccine is especially urgent with respect to preventing infection by certain bacteria, viruses and parasites. Some bacteria and especially certain viruses mutate constantly or mutate when passing through an intermediate host, often rendering the vaccine developed to the prior or originating bacteria or virus useless against the new strains that emerge. As a consequence, some vaccines may need to be reformulated yearly (or more often) and often administered at fairly high doses. The development and manufacturing costs are high and administering vaccines pose a great many complications and associated risks to patients.

Immunological compositions, vaccines, antigens, and epitopes as disclosed herein, including specific combination as described herein, were surprisingly discovered to treat and/or prevent neurodegenerative disorders, to reduce the inflammatory response, and/or to treat sepsis. These antigens, preferably produced recombinantly or otherwise synthetically, contain or are derived from a plurality of antigenic regions (e.g., epitopes which may be continuous or discontinuous epitopes) of a pathogen or of different pathogens. Without being limited to theory, it is believed that the accumulated toxins in the systems of infected individuals, often due to sepsis, crosses the blood/brain barrier and kills or debilitates brain cells, leading to neurodegenerative disorders such as but not limited to Alzheimer's disease, and to the initiation and/or exacerbation of the inflammation response which can become chronic and lead to disorder related to chronic inflammation.

Antigens as described herein, which may contain an antigenic region or one or more molecules of the microorganism including pathogenic microorganisms that represent a combination of all or parts of two or more epitopes (e.g., a composite peptide), or a plurality of immunologically responsive regions derived from one or multiple antigenic sources (e.g., epitopes of viruses, parasites, bacteria, fungi, cells). These immunological regions are amino acid sequences or epitopes that are generally highly conserved sequences (e.g., sequences in common between different serotypes, subspecies and/or species) found at those antigenic regions of a pathogen or other antigen associated with an infection or a disease or, importantly, associated with stimulation of the immune system to provide protection against the pathogen. For administration to humans, vaccines and/or immunological compositions disclosed herein may be administered via injection (e.g., intramuscular, intradermal, intravenous, intraperitoneal) or taken orally or intranasally. For administration to animals, preferably, immunogenic compositions are administered collectively such as in a water or food supply, or as an aerosol dispensed in a closed or partially closed environment, thereby avoiding the need and expense of providing the vaccine individually.

Epitope vaccine antigen sequences are unique peptide antigens that combine conserved peptide sequences from the same, or different microbes into one sequence that provides a peptide that is different from any peptide sequence found in nature. Peptide epitopes may be known or previously unknown epitopes that have been identified in microbes such as bacteria, parasites, fungi, or viruses. One or more epitopes from a single microbe can be sequenced as a single, or repeated epitope and may be combined with one or more epitopes from one or more other pathogens in a continuous peptide sequence. The peptide antigens may be to a single microbe or to one or more microbes, or viruses, such as for example, influenza, coronavirus, adenovirus, and respiratory syncytial virus. The peptide antigen may also be from a single bacterium, or from one or more gram positive, or gram-negative bacteria, such Pneumococcus spp., Staphylococcus spp. (e.g., S. aureus), Mycobacteria spp. (e.g., M. tuberculosis, M. smegmatis, M. leprae, M. kansasii, M. mantenii, M. fortuitum, or M. xenopi), Bacillus spp. (e.g., B. subtilis), Escherichia spp. (e.g., E. coli), Haemophilus spp. (e.g., H. influenza), Salmonella spp., etc. The epitopes may be combined in any order or configured to provide an immunogenic structure that induces an immune response in a host immunized with the peptide vaccine.

One embodiment of the invention is directed to one or more peptides, preferably produced recombinantly or otherwise synthetically, of pathogenic microorganisms such as, for example, PGN, LTA, mycolic acid, and LPS molecules of one of more microbes. Antigens and peptide epitopes disclosed herein may be selected regions of the respective protein that are known or believed to generate an effective immune response after administration that eliminates and/or reduces the presence of pathogenic microorganisms. The peptide sequence may contain a plurality of immunologically responsive regions or epitopes. The peptide sequence may be a composite of the epitopes found within a protein or two or more proteins, which can be artificially arranged, although preferably along a single amino acid sequence or peptide. The plurality may contain multiples of the same epitope, although generally not in a naturally occurring order, or multiples of a variety of different epitopes from one or more pathogens. Epitopes are conformational sites on a microbial antigen where an antibody binds, typically characterized by 3D surface features. A conformational epitope is an epitope on a microbe, which may be a synthetic peptide, wherein the 3D structure of both the microbial peptide epitope and the synthetic peptide epitope are the same as demonstrated by the binding of an antibody to both the epitope on the microbe and the synthetic peptide epitope. Conformational epitopes may be non-linear or linear structures. A conformational structure that mimics the 3D structure of a microbial epitope wherein the antibody to the mimotope binds to the epitope on the microbial antigen and antibody to the microbial epitope binds to the mimotope

Epitopes may be identical to known immunological regions of a pathogen, mimotopes of known immunological regions of a pathogen (mimotopes are distinguished from epitopes in that mimotopes do not have epitope components, but retain the 3D structure of the epitope wherein the antibody to the mimotope binds to the epitope and antibody to the microbial epitope binds to the synthetic mimotope. Also, mimotopes may contain a slightly different sequence or entirely new construct that has not previously existed and therefore artificially constructed. Preferably, the antigen of this disclosure induces a protective immunogenic response in the animal or a mammal (e.g., human) and stimulates both mucosal and systemic immune responses similar to those of the natural infection. Preferably that response includes the production of killer T-cell (T_{C} or CTL) responses, helper T-cell (T_{H}) responses, macrophages (MP), and specific antibody production in an inoculated subject. Also preferably the response generated in a mammal is opsonic such that neutrophils and macrophages are invoked that are able to phagocytize and kill pathogens and harmful microbes.

Preferably, the one or more peptides are an immunologically responsive composition of multiple peptides with epitopes of multiple pathogenic microbes. Administration of the immunogenic composition stimulates the immune system of the host to generate an immunological response to the multiple microbes, thus clearing the microbes or significantly reducing their number to ameliorate the disease or disorder. Preferably, the immunogenic composition contains 2 or more peptides, 3 or more peptides, 3 or more peptides, 4 or more peptides, 5 or more peptides, 6 or more peptides, 7 or more peptides, 8 or more peptides, 9 or more peptides, or 10 or more peptides. Preferably different peptides are contained within one or more contiguous sequences.

Antigens of the invention may also be obtained or derived from the sequences of a pathogen such as, for example, multiple or combined epitopes of the molecules, proteins, and/or polypeptides of gram-positive and/or gram-negative bacteria, for example, but not limited to Streptococcus, Pseudomonas, Mycobacterium such as M. tuberculosis, Shigella, Campylobacter, Salmonella, Haemophilus influenza, Chlamydophila pneumonia, Corynebacterium diphtheriae, Clostridium tetani, Mycoplasma pneumonia, Staphylococcus aureus, Moraxella catarrhalis, Legionella pneumophila, Bordetella pertussis, Escherichia coli, such as E. coli 0157, and multiple or combined epitomes of conserved regions of any of the foregoing. Exemplary parasites from which sequences may be obtained or derived include but are not limited to Plasmodium such as Plasmodium falciparum and Trypanosoma. Exemplary fungi include, but are not limited to, Aspergillus fumigatus and Aspergillus flavus. Exemplary viruses include, but are not limited to arena viruses, bunyaviruses, coronaviruses, paramyxoviruses, filoviruses, Hepadna viruses, herpes viruses, orthomyxoviruses, orthopneumovirus, parvoviruses, picornaviruses, papillomaviruses, reoviruses, retroviruses, rhabdoviruses, and togaviruses. Preferably, the virus epitopes are obtained or derived from sequences of Influenza viruses.

Antigens as disclosed herein include antigens which are engineered, artificially created antigens made from two or more epitopes, such that the resulting antigen has physical and/or chemical properties that differ from or are additive of the individual epitopes. Preferable the antigen, when exposed to the immune system of a mammal or an animal, is capable of simultaneously generating an immunological response to each of the epitope and preferably to a greater degree (e.g., as measurable from a cellular or humoral response to an identified pathogen) than the individual epitopes. In addition, the antigen provides the added function of generating a protective immunological response in a mammal or an animal when used as a vaccine and against each of the constituent epitopes. Preferably, the epitope or antigen has the additional function of providing protection against not only the pathogens from which the constituents were derived, but related pathogens as well. These related pathogenic organisms may be different strains and/or different serotypes of the same species of organism, or different species of the same genus of organism, or different organisms entirely that are only related by a common epitope.

Peptides may contain one or more epitopes that represent two or more epitopes with epitope sequences only similar to the epitope sequences from which they were derived. Epitopes are regions obtained or derived from a conserved region of a protein or peptide of a pathogen that elicit a robust immunological response when administered to a mammal or an animal. Preferably, that robust response provides the subject with an immunological protection against developing disease from exposure to the pathogen. A preferred example is an epitope, which is one artificially created from a combination of two or more highly conserved, although not identical, amino acid sequences of two or more different, but otherwise related pathogens. The pathogens may be of the same type, but of a different strain, serotype, or species or other relation. The epitope contains the conserved region that is in common between the related epitopes, and also contains the variable regions which differ. The sequences of a epitope that represents a combination of two conserved, but not identical sequences. Preferably the conserved region contains about 20 or less amino acids on each side of the variable amino acids, preferably about 15 or less, preferably about 10 or less, preferably about 8 or less, preferably about 6 or less, and more preferably about 4 or less. Preferably the amino acids that vary between two similar, but not identical conserved regions are 5 or less, preferably 4 or less, preferably 3 or less, preferably 2 or less, and more preferably only one.

A “composite epitope,” similar to the composite antigen, is an engineered, artificially created single epitope made from two or more constituent epitopes, such that the resulting composite epitope has physical and/or chemical properties that differ from or are additive of the constituent epitopes. Preferably, the composite epitope, when exposed to the immune system of a mammal or an animal, is capable of simultaneously generating an immunological response to each of the constituent epitopes of the composite and preferably to a greater degree than that achieved by either of the constituent epitopes individually. In addition, the composite epitope provides the added function of generating a protective immunological response in a patient when used as a vaccine and against each of the constituent epitopes. Preferably, the composite has the additional function of providing protection against not only the pathogens from which the constituents were derived, but related pathogens as well. These related pathogenic organisms may be strains or serotypes of the same species of organism, or different species of the same genus of organism, or different organisms entirely that are only related by a common epitope.

Epitopes of the invention are entirely artificial peptide molecules that do not otherwise exist in nature and to which an immune system has not been otherwise exposed. Preferably, these conserved immunological regions that are combined as a epitope represent immunologically responsive regions of proteins and/or polypeptides that are highly conserved between related pathogens. Although a vaccine can be developed from a single epitope, in many instances the most effective vaccine may be developed from multiple, different epitopes.

Antigens of the invention may contain one or more epitopes, which may include one or more known epitopes to provide an effective vaccine. Although antigens may comprise a single epitope, an antigen may not comprise only a single known epitope. Preferably, the immunological response achieved from a vaccination with an antigen, or group of epitopes antigens, provides protection against infection caused by the original strains from which the sequence of the antigen was derived and also provides immunological protection against other strains, serotypes and/or species that share one or more of the general conserved regions represented in the antigen. Preferably that response stimulates both mucosal and systemic immune responses in the mammal or the animal, similar to those of the natural infection. Thus, the resulting immune response achieved from a vaccination with an antigen is more broadly protective than can be achieved from a conventional single antigen vaccination against multiple strains, serotypes, and species or otherwise related pathogens regardless of antigenic drift that may take place in the evolution of the pathogen. Preferably, vaccines developed from antigens of the invention avoid any need for repeated or annual vaccinations, the associated complications and expenses of manufacture, and the elevated risks to the subject. These vaccines are useful to treat individual animals, mammals, and populations or either, thereby preventing infection and mortality and subsequently infections in mammals including pandemics. Such vaccines are also useful to compliment conventional vaccines.

As discussed herein, the antigen preferably comprises a single chain of amino acids with a sequence derived from one or more epitopes or a plurality of epitopes, that may be the same or different, and is preferably produced recombinantly or otherwise synthetically. Epitope sequences may be repeated consecutively and uninterrupted along a sequence or interspersed among other sequences that may be single or a few amino acids as spacers or sequences that encode peptides (collectively spacers), and may be nonimmunogenic or immunogenic and capable of inducing a cellular (T cell) or humoral (B cell) immune response in an animal or a mammal. T-cell stimulating antigens include, for example, tetanus toxin, tetanus toxin heavy chain proteins, diphtheria toxoid (e.g., recombinantly engineered or purified CRM197), tetanus toxoid, Pseudomonas exoprotein A, Pseudomonas aeruginosa toxoid, Bordetella pertussis toxoid, Clostridium perfringens toxoid, Escherichia coli heat-labile toxin B subunit, Neisseria meningitidis outer membrane complex, Hemophilus influenzae protein D, Flagellin Fli C, Horseshoe crab Haemocyanin, and fragments, derivatives, and modifications thereof. Peptides sequence from unrelated microbes may be combined into a single antigen. For example, viral sequences of selected immunoresponsive peptides may be interspersed with conserved sequences or epitopes selected from other microbes, such as, for example, bacteria such as M. tuberculosis, S. pneumococcus, P. aeruginosa or S. aureus, viruses such as respiratory viruses, or parasites, such as malaria. Preferred viral proteins, from which preferred epitopes may be selected, include, but are not limited to the influenza virus proteins HA, NA, and M2e, and/or coronavirus proteins spike(S), polymerase (POL), envelope (E), membrane (M), and nucleocapsid (N).

An epitope of an antigen disclosed herein may be of any sequence and size, but is preferable composed of natural amino acids or mimotopes (i.e., a peptide and mimics the structure of an epitope but is composed of a different amino acid sequence than the natural epitope) and is more than 5 but less than 100 amino acids in length, preferably less than 80, preferably less than 70, preferably less than 60, preferably less than 50, preferably less than 40, preferably less than 30, preferably between 5 and 25 amino acids in length, preferably between 8 and 20 amino acids in length, and more preferably between 5 and 15 amino acids in length. Mimotopes may have 1, 2, 3, 4, 5, 6, 7, 8, 9, 10 or more amino acid differences as compared to the natural epitope. Antigens preferably contain any number of epitopes. The most effective number of epitopes of an antigen against a particular pathogen, pathogen family, or group of pathogens may be determined by one skilled in the art from the disclosures of this application and using routine testing procedures. Antigens may be effective with one epitope, preferably with 2 or more, 3 or more 4 or more, 5 or more or greater. Optionally, antigens may include one or more spacers between epitopes which may be sequences of antigenic regions derived from the same or from one or more different pathogens, or sequences that serve as immunological primers or that otherwise provide a boost to the immune system. That boost may be generated from a sequence of amino acids that are known to stimulate the immune system, either directly or as an adjuvant. Preferred added adjuvants comprise toll-like receptor agonists, analgesic adjuvants, inorganic compounds such as alum, aluminum hydroxide, oil in water emulsion, squalene oil in water nano-emulsion, aluminum phosphate, calcium phosphate hydroxide, mineral oil such as paraffin oil, bacterial products such as killed bacteria Bordetella pertussis, Mycobacterium bovis, toxoids, nonbacterial organics such as squalene, detergents, plant saponins such as Quillaja (Quil A), soybean, Polygala senega, cytokines such as IL-1, IL-2, IL-12, Freund's complete adjuvant, Freund's incomplete adjuvant, food-based oil, Adjuvant 65, which is a product based on peanut oil, and derivatives, modifications and combinations thereof. Preferred adjuvants include, for example, AS01 (Adjuvant System 01) which comprises TLR4 ligand, 3-O-desacyl-4′-monophosphoryl lipid (MPL), and a saponin, QS-21, AS01b which is a component of the adjuvant Shingrix, ALF (Army Liposome Formulation) which comprises liposomes containing saturated phospholipids, cholesterol, and/or monophosphoryl lipid A (MPLA) as an immunostimulant. ALF has a safety and a strong potency. ALF modifications and derivatives include, for example, ALF adsorbed to aluminum hydroxide (ALFA), ALF containing QS21 saponin (ALFQ), and ALFQ adsorbed to aluminum hydroxide (ALFQA). A preferred added adjuvant formulation comprises a liposome, saponin, lipid A, squalene, unilamellar liposomes having a liposome bilayer that comprises at least one phosphatidylcholine (PC) and/or phosphatidylglycerol (PG), as phospholipids, which may be dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and/or distearyl phosphatidylglycerol (DSPG), a cholesterol, a monophosphoryl lipid A (MPLA), and a saponin. Preferably the mole ratio of the cholesterol to the phospholipids is greater than about 50:50, and also that the unilamellar liposomes have a median diameter size in micrometer range as detected by light scattering analysis. Additional preferred adjuvants are disclosed in U.S. Pat. No. 10,434,167, which issued Oct. 8, 2019, the entirety of which is incorporated by reference herein.

Another form of antigen, preferably produced recombinantly or otherwise synthetically, comprises a contiguous sequence of one or more epitopes, which may comprise known epitopes, from one or more pathogens in a sequence that does not exist naturally and must be artificially constructed. For example, a contiguous sequence may contain epitopes in closer proximity to each other than would otherwise occur naturally or may contain spacer sequences between epitopes that do not otherwise occur naturally. Preferably, a contiguous sequence of the invention contains one or more epitopes, which is a combination of the sequences of the conserved regions of epitopes that are common to multiple pathogens plus those amino acids that differ between the two conserved regions. For example, where two pathogens contain similar conserved regions that differ by only a single amino acid, the sequences would include the conserved region amino acids and each of the amino acids that differ between the two regions as discussed herein.

It is also preferable that an antigen of the invention contains a plurality of repeated epitopes and, optionally, epitopes conjugated with linker regions between or surrounding each epitope, and the plurality of epitopes be the same or different. Preferred linkers include amino acid sequences of antigenic regions of the same or of different pathogens, or amino acids sequences that aid in the generation of an immune response. Preferred examples include, but are not limited to, any of the various antigenic regions of bacteria such as, but not limited to M. tuberculosis, S. aureus and E. coli and viruses such as, but not limited to influenza, coronavirus and HIV and parasites such as P. falciparum. It is also preferred that antigens contain epitopes that generate a systemic and/or a mucosal immune responses similar to that produced from a natural infection.

Another embodiment of the invention is directed to methods for treating or preventing viral or bacterial infections and the conditions caused by the infections or bacterial toxins to include sepsis, shock, and neurodegenerative disorders in a mammal comprising administering to the mammal bifunctional, polyclonal, and/or monoclonal antibodies that are specifically reactive against the peptides disclosed here. Preferably the polyclonal, bifunctional, or monoclonal antibodies generate viral neutralizing, opsono-phagocytic activity, destruction of the microorganism, enhanced cytokine induced immunity to the microorganism and/or neutralizes toxic substances of the microorganism, and/or cocktails of two or more (2, 3, 4, 5, 6, 7, or more) monoclonal antibodies (MABs) that enhance immunity to the microorganism and/or neutralize viruses and toxins. Preferably, the antibodies are polyclonal antibodies or monoclonal antibodies and react against one or more of the target proteins and the MABS have extended half-life.

Another embodiment of the invention is directed to methods for the treatment of various forms of cancer including pancreatic, ovarian, prostate, non-small cell cancers, breast, and lung cancers, and also inflammation and its related diseases and disorders. Methods involve the administration of compositions as disclosed herein such as collections of peptides containing epitopes of one or more of the molecules of peptidoglycan (PGN), lipoteichoic acid (LTA), lipopolysaccharide (LPS), deoxycholic acid (DCA), and/or compositions of antibodies that are specifically reactive to the one or more of these epitopes. Preferably, the antibodies provide an opsonophagocytic response. Preferably the composition and/or to reduce the proliferation of cancer cells, and may be used alone or together with conventional treatments options for these disorders. Method involved administering the composition disclosed herein to a subject, which is preferably an adult, a child, or an infant, or a human male or a human female. Treatments may be administered or supplemented with compositions that provoke the immune system to respond to the overexpression of normal cytokines (e.g., a group of small proteins important in cell signaling), such as the chemokines, interferons, interleukins, lymphokines, tumor necrosis factors, CD14 (cluster of differentiation factor 14), NFkB (Nuclear factor kappa-light-chain-enhancer of activated B cells), MAPK (mitogen-activated protein kinase), growth factors such as vascular endothelial growth factor (VEGF), prostaglandins such as prostaglandin E2 (PGE2), and other proinflammatory factors. These molecules can be overly present in the system and contribute to the development of cancer and the inflammatory response. Compositions comprised of collections of multiple epitopes of these molecules and/or collections of antibodies directed against these molecules may be administered. Preferably, administration of a composition as disclosed herein prevents the cascade of proliferation inducing agents and shuts down development of cancer and/or inflammation. Preferably, administration is oral, sub-cutaneous, intra-muscular, intradermal, or intra-nasal.

Another embodiment of the invention is directed to antibodies that are specifically reactive against epitopes of the microorganism. Preferably the antibody is a monoclonal antibody and an IgA, IgD, IgE, IgG or IgM (including subtypes thereof such as, for example, IgG1, IgG2, IgG2A, IgG2B, IgG2C, IgG3 and IgG4), and may be derived from most any mammal such as, for example, human, porcine, caprine, murine, leporidae, muridae, and equine, to include rabbit, guinea pig, mouse, human, fully or partly humanized, chimeric or single chain of any of the above. Antibodies specific for peptides of the invention can be generated by methods well known in the art. Such antibodies can include, but are not limited to, polyclonal, monoclonal, chimeric, humanized, single chain, peptides of antibody fragments, Fab fragments and fragments produced by an Fab expression library. These antibodies alone or in combination other antibodies or agents against a pathogen specifically target and neutralize the pathogenic microorganism. Extended half-life antibodies (monoclonal or polyclonal) can be formed that offer sustained protection by remaining in circulation for extended periods. Modifications to extend circulating half lives are preferably through recombinant engineering such as YTE modifications. A YTE-modified antibody is an engineered antibody with specific mutations in its Fc region (Y252T, S254T, and T256E) that increases its binding affinity to the neonatal Fc receptor (FcRn), which recycles antibodies back into the bloodstream instead of degrading them and other techniques to extend half-life can be used. Numerous methods for producing polyclonal and monoclonal antibodies are known to those of skill in the art, and can be adapted to produce antibodies specific for the polypeptides of the invention, and/or encoded by the polynucleotide sequences of the invention (see, e.g., Coligan Current Protocols in Immunology Wiley/Greene, NY; Paul (ed.) (1991); (1998) Fundamental Immunology Fourth Edition, Lippincott-Raven, Lippincott Williams & Wilkins; Harlow and Lane (1989) Antibodies: A Laboratory Manual, Cold Spring Harbor Press, NY, USA; Stites et al. (Eds.) Basic and Clinical Immunology (4th ed.) Lange Medical Publications, Los Altos, CA, USA and references cited therein; Goding, Monoclonal Antibodies: Principles and Practice (2d ed.) Academic Press, New York, NY, USA; 1986; and Kohler and Milstein (1975).

The DNA encoding the antibodies may be utilized in any appropriate recombinant cell line to produce the encoded MABs. Another embodiment comprises hybridoma cultures that produce the monoclonal antibodies or antibody parts. Another embodiment of the invention comprises non-naturally occurring polyclonal antibodies that are specifically reactive against the microorganism. Some important monoclonal antibodies are described in Table 1.

TABLE 1

| | | | Hybridoma | Hybridoma | Target | mAb |
|---|---|---|---|---|---|---|
| Mouse ID | Immunogen | Conjugate | (mAb) ID | Clone ID | Antigen | Isotype |
| MS 1435 | TB Pep01 | CRM | LD7 | LD7 I BB2 I B9 | 16 kD | IgG2a |
| | | | | | HSP16.3 | |
| | | | | | TB Pep01 | |
| MS 1435 | TB Pep01 | CRM | CA6 | CA6 II GA8 I A5 | 16 kD | IgG2b |
| | | | | | HSP16.3 | |
| | | | | | TB Pep01 | |
| MS 190 | Ultrapure | CRM | MD11 | MD11 I C11 | PGN | IgG2b |
| | Peptidoglycan | | | | | |
| | from S. aureus | | | | | |
| | (PGN) | | | | | |
| MS 2209 | A/Wuhan (H3N2) + | CRM | NB5 | NB5 II C2 I K8 | Neuraminidase | IgG2a |
| | Flu Pep11 | | | | (NA) Flu | |
| | | | | | Pep10 | |
| MS 2209 | A/Wuhan (H3N2) + | CRM | LD9 | LD9 III D6 | Hemagglutinin | IgG1 |
| | Flu Pep11 | | | | (HA) Flu | |
| | | | | | Pep06 | |
| MS 2209 | A/Wuhan (H3N2) + | CRM | EA9 | EA9 I F7 | Hemagglutinin | IgG1 |
| | Flu Pep11 | | | | (HA) Flu | |
| | | | | | Pep03 | |
| MS 1443 | Flu Pep5906 | CRM | GA4 | GA4 I G11 | Matrix | IgG1 |
| | | | | | (M1/M2/M2e) | |
| | | | | | Flu Pep5906 | |
| MS 2016 | Flu Pep5906 + Flu | CRM | CG6 | CG6 II H8 | Matrix | IgG3 |
| | Pep11 | | | | (M1/M2/M2e) | |
| | | | | | Flu Pep5906 | |
| MS 2016 | Flu Pep5906 + Flu | CRM | KC7 | KC7 I D8 | Matrix | IgG3 |
| | Pep11 | | | | (M1/M2/M2e) | |
| | | | | | Flu Pep5906 | |
| DRAGA5 | Ultrapure | CRM | DRG-5 | DRG-5 BD11 II | PGN | IgM |
| | Peptidoglycan | | BD11 | E6 II G1 | | |
| | from S. aureus | | | | | |
| | (PGN) + TB | | | | | |
| | Pep01 | | | | | |
| MS 1323 | EtOH-K MTB | Not | JG7 | JG7 III D3 I F9 | PGN MTB | IgG1 |
| | | applicable | | | | |
| MS1420 | EtOH-K MTB | Not | GG9 | GG9 II G2 | MTB | IgG1 |
| | | applicable | | | | |
| MS1420 | EtOH-K MTB | Not | AB9 | AB9 I A5 | MTB | IgG1 |
| | | applicable | | | | |

Hybridoma cell lines that express the monoclonal antibodies disclosed herein were deposited with the American Type Culture Collection (ATCC; Manassas, VA). Hybridomas that produce monoclonal antibodies EA9 (PTA-127659), KC7 (PTA-127660), DRG-5 BD11 (PTA-127658), CG6 (PTA-127661), and LD9 (PTA-127662) (as identified in Table 1) were each deposited with ATCC on Oct. 13, 2023. Hybridomas that produce monoclonal antibodies MD11 (PTA-127712), GA4 (PTA-127713), and NB5 (PTA-127714), (as identified in Table 1) were each deposited with ATCC on Mar. 13, 2024. Hybridomas that produce monoclonal antibodies JG7 (PTA-124416), GG9 (PTA-124417), and A9 (PTA-124418), (as identified in Table 1) were each deposited with ATCC on Aug. 17, 2017. Monoclonal antibodies produced by these hybridomas may include variable and hypervariable regions, CDR, and Fc regions that may be separately obtained and useful as such. These monoclonal antibodies may be fully or partly humanized, bispecific, have extended half-life, and/or conjugated with, for example, molecules targeted against a particular pathogen. Another embodiment of the invention is directed to methods for treating or preventing infection by administering a monoclonal, multiple monoclonal, or polyclonal antibody that is specifically reactive against a microorganism. These antibodies may be coupled with other agents that target the pathogen. Coupling may be covalent, such as via conjugation (e.g., with bacterial or viral polysaccharides) or non-covalent, or the molecules may be co-administered.

Another embodiment of the invention is directed to method of immunizing mammals or animals with the immunogenic compositions of the invention. Although these immunogenic composition and/or vaccines of the invention preferably do not require repeated administration to maintain protection, two or more or annual administration may be necessary or desired. In addition, the compositions and vaccines of the invention generally and advantageously provide increased safety considerations, both in their manufacture and administration (due in part to a substantially decreased need for repeated administration), a relatively long shelf life in part due to minimized need to reformulate due to strain-specific shift and drift, an ability to target immune responses with high specificity for particular microbial epitopes, and an ability to prepare a single vaccine that is effective against multiple pathogens, each of which may be a different.

The invention encompasses antigenic compositions, preferably produced recombinantly or otherwise synthetically, methods of making such compositions, and methods for their use in the prevention, treatment, management, and/or prophylaxis of an infection. The compositions disclosed herein, as well as methods employing them, find particular use in the treatment or prevention of viral, bacterial, parasitic and/or fungal pathogenesis and infection using immunogenic compositions and methods superior to conventional treatments presently available in the art. Preferably, vaccinations of immunogenic compositions of antigens disclosed herein provide protection against a pathogenic infection and neural degenerative disorders for more than a one-year cycle, which is typical for pathogens such as influenza virus. More preferably, protection is provided for up to 2 years, 5 years, 10 years, 15 years, 20 years, or longer.

Another embodiment of the invention is directed to an immunogenic composition comprising nucleic acid sequences that encode protective antigens that contain epitopes against LPS, LTA, and PGN. The sequences can be incorporated into a viral vector, suitable for immunizing a mammal.

Peptides or polypeptides of the invention includes at least two conserved epitope sequences, preferably three, which may also comprise one or more repeats of the same or a different epitope sequence, each of which is conserved across a plurality of homologous proteins. In exemplary antigens, at least one epitope sequence (continuous or discontinuous) may be repeated at least once or multiple times. Preferably the compositions of the invention include a pharmaceutically acceptable carrier.

Compositions of the invention may include one or more T-cell stimulating epitopes, such as epitopes from diphtheria toxoid, tetanus toxoid, a polysaccharide, a lipoprotein, or a derivative or any combination thereof (including fragments or variants thereof). Typically, the at least one repeated sequence of the antigen is contained within the same molecule as the T-cell stimulating epitopes. In the case of protein-based T-cell stimulating epitopes, the at least one repeated sequence of the antigen may be contained within the same polypeptide as the T-cell stimulating epitopes, may be conjugated thereto, or may be associated in other ways. Preferably, one or more T-cell stimulating epitopes are positioned at either the N-Terminus or the C-Terminus (or both) of the antigen.

In additional embodiments, the compositions of the invention, with or without associated T-cell stimulating epitopes, may include one or more epitopes of a heat shock protein (e.g., an alpha helix portion), or a lipoarabinomannan protein (LAM). Preferably, the composition includes an adjuvant which is preferably a toll-like receptor agonist and/or nano-emulsion.

Antigens of the invention may be synthesized by in vitro chemical synthesis, solid-phase protein synthesis, and in vitro (cell-free) protein translation, or recombinantly engineered and expressed in bacterial cells, fungi, insect cells, mammalian cells, virus particles, yeast, and the like.

The invention encompasses methods of preparing an immunogenic composition, preferably a pharmaceutical composition, more preferably a vaccine, wherein a target antigen of the present invention is associated with a pharmaceutically acceptable diluent, excipient, or carrier, and may be used with most any adjuvant, such as, for example, ALFQ, ALFQA, ALFA, AS01, AS01b, and/or combinations, derivatives, and modifications thereof.

Within the context of the present invention, that a relatively small number of conservative or neutral substitutions (e.g., 1 or 2) may be made within the sequence of the antigen or epitope sequences disclosed herein, without substantially altering the immunological response to the peptide. In some cases, the substitution of one or more amino acids in a particular peptide may in fact serve to enhance or otherwise improve the ability of the peptide to elicit a systemic response in an animal or a mammal that has been provided with a composition that comprises the modified peptide, or a polynucleotide that encodes the peptide. Suitable substitutions may generally be identified using computer programs and the effect of such substitutions may be confirmed based on the reactivity of the modified peptide with antisera and/or T-cells. Accordingly, within certain preferred embodiments, a peptide for use in the disclosed diagnostic and therapeutic methods may comprise a primary amino acid sequence in which one or more amino acid residues are substituted by one or more replacement amino acids, such that the ability of the modified peptide to react with antigen-specific antisera and/or T-cell lines or clones is not significantly less than that for the unmodified peptide.

As described above, preferred peptide variants are those that contain one or more conservative substitutions. A “conservative substitution” is one in which an amino acid is substituted for another amino acid that has similar properties, such that one skilled in the art of peptide chemistry would expect the secondary structure and hydropathic nature of the peptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Examples of amino acid substitutions that represent a conservative change include: (1) replacement of one or more Ala, Pro, Gly, Glu, Asp, Gln, Asn, Ser, or Thr; residues with one or more residues from the same group; (2) replacement of one or more Cys, Ser, Tyr, or Thr residues with one or more residues from the same group; (3) replacement of one or more Val, Ile, Leu, Met, Ala, or Phe residues with one or more residues from the same group; (4) replacement of one or more Lys, Arg, or His residues with one or more residues from the same group; and (5) replacement of one or more Phe, Tyr, Trp, or His residues with one or more residues from the same group. A variant may also, or alternatively, contain non-conservative changes, for example, by substituting one of the amino acid residues from group (1) with an amino acid residue from group (2), group (3), group (4), or group (5). Variants may also (or alternatively) be modified by, for example, the deletion or addition of amino acids that have minimal influence on the immunogenicity, secondary structure and hydropathic nature of the peptide.

Epitopes may be arranged in any order relative to one another in the sequence which may be with or without spacers. The number of spacer amino acids between two or more of the epitopic sequences can be of any practical range, including, for example, from 1 or 2 amino acids to 3, 4, 5, 6, 7, 8, 9, or even 10 or more amino acids between adjacent epitopes.

Another embodiment of the invention is directed to polynucleotides including DNA, RNA (e.g., cRNA, mRNA), and PNA (peptide nucleic acid) constructs that encode the sequences of the invention. These polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials. As is appreciated by those of ordinary skill in the art that, as a result of the degeneracy of the genetic code, there are many nucleotide sequences that encode a given primary amino acid sequence. Some of these polynucleotides bear minimal homology to the nucleotide sequence of any native gene. Nonetheless, polynucleotides that vary due to differences in codon usage are specifically contemplated by the present invention. Polynucleotides that encode an immunogenic peptide may generally be used for production of the peptide, in vitro or in vivo. Any polynucleotide may be further modified to increase stability in vivo. Possible modifications include, but are not limited to, the addition of flanking sequences at the 5′ and/or 3′-ends; the use of phosphorothioate or 2′-o-methyl rather than phosphodiesterase linkages in the backbone; and/or the inclusion of nontraditional bases such as inosine, queosine and wybutosine, as well as acetyl-methyl-, thio- and other modified forms of adenine, cytidine, guanine, thymine and uridine.

A nucleic acid vaccine of the invention contains the genetic sequence of an antigen as CRNA or mRNA, or DNA, plus other necessary sequences that provide for the expression of the antigen in cells. Nucleic acid vaccines may also contain the genetic sequence of antibodies or parts of antibodies to be produced in the subject. By injecting the mammal with genetically engineered nucleic acid antigen vaccines, the antigen is produced in or preferably on cells, which the mammal's immune system recognizes and thereby generates a humoral or cellular response to the antigen, and therefore the pathogen. By injecting the mammal with genetically engineered nucleic acid antibody vaccines, the antibody is produced in the subject, recognizes the target pathogen, and thereby directly generates a response to the pathogen. Nucleic acid vaccines have a number of advantages over conventional vaccines, including the ability to induce a more general and complete immune response in the mammal. Accordingly, nucleic acid vaccines can be used to protect an animal or a mammal against disease caused from many different pathogenic organisms of viral, bacterial, and parasitic origin as well as certain tumors.

Nucleic acid vaccines typically comprise a viral or bacterial nucleic acid (e.g., cRNA, mRNA, DNA) that encodes an antigen contained in vectors or plasmids that have been genetically modified to transcribe and translate the antigenic sequences into specific protein sequences derived from a pathogen. By way of example, the nucleic acid vaccine is administered, and the cellular machinery transcribed and/or translates the nucleic acid into the antigens which produce an immune response. The antigens, being non-natural and unrecognized by the mammalian immune system, are processed by cells and the processed proteins, preferably the epitopes, displayed on cell surfaces. Upon recognition of these antigens as foreign, the immune system generates an appropriate immune response that protects from the infection. In addition, nucleic acid vaccines of the invention are preferably codon optimized for expression in the animal (or mammal) of interest. In a preferred embodiment, codon optimization involves selecting a desired codon usage bias (the frequency of occurrence of synonymous codons in coding DNA) for the particular cell type so that the desired peptide sequence is expressed.

Compositions of the invention may contain antigens of epitope sequences, and/or RNA and/or DNA vaccines that encode such sequences. Composition may include adjuvants such as, for example, oil in water emulsion, ALFQ, ALFQA, ALFA, AS01, AS01b, and/or combinations, derivatives, and modifications thereof. The formulation of pharmaceutically-acceptable excipients and carrier solutions is well known to those of ordinary skill in the art, as is the development of suitable dosing and treatment regimens for using the particular compositions described herein in a variety of treatment regimens.

The amount of immunogenic composition(s) and the time needed for the administration of such immunogenic composition(s) will be within the purview of the ordinary-skilled artisan having benefit of the present teachings. The administration of a therapeutically-effective, pharmaceutically-effective, and/or prophylactically-effective amount of the disclosed immunogenic compositions may be achieved by a single administration. Alternatively, in some circumstances, it may be desirable to provide multiple, or successive administrations of the immunogenic compositions, either over a relatively short, or even a relatively prolonged period of time, as may be determined by the skilled person overseeing the administration of such compositions.

The immunogenic compositions and vaccines of the present invention preferably contain an adjuvant such as oil in water emulsion or ALFQ and may be given by IM, SQ, Intradermal or intranasal administration or in a manner compatible with the dosage formulation, and in such an amount as will be prophylactically or therapeutically effective and preferably immunogenic. The quantity to be administered depends on the subject to be treated, including, e.g., the capacity of the immune system to mount an immune response, and the degree of protection desired. Suitable dosage ranges may be on the order of several hundred micrograms (μg) of active ingredient per animal or mammal with a preferred range from about 0.1 μg to 2,000 μg dry weights (even though higher amounts, such as, e.g., in the range of about 1 to about 10 mg are also contemplated), such as in the range from about 0.5 μg to 1,000 μg, preferably in the range from about 1 μg to about 500 μg and especially in the range from about 10 μg to about 100 μg. Suitable regimens for initial administration and booster shots are also variable but are typified by an initial administration followed by optional but preferred subsequent inoculations or other periodic administrations.

An effective dose comprises an amount in the range of about 0.1 μg to about 1 mg total protein or target antigen (dry weight) per animal or mammal. However, one may prefer to adjust dosage based on the amount of peptide delivered. In either case, these ranges are merely guidelines from which one of ordinary skill in the art may deviate according to conventional dosing techniques. Precise dosages may be determined by assessing the immunogenicity of the conjugate produced in the appropriate host so that an immunologically effective dose is delivered. An immunologically effective dose is one that stimulates the immune system of the animal or mammal to establish an immune response to the immunogenic composition or vaccine. Preferably, a level of immunological memory sufficient to provide long-term protection against neurological degeneration is obtained. The immunogenic compositions or vaccines of the invention may be preferably formulated with an adjuvant. By “long-term” it is preferably meant over a period of time of at least about 6 months, over at least about 1 year, over at least about 2 to 5 or even at least about 2 to about 10 years or longer. Preferably protection is provided with one administration (or one initial series of administrations) and multiple administrations over time are not required.

The following examples illustrate embodiments of the invention but are not to be viewed as limiting the scope of the invention.

### EXAMPLES

### Example 1 Peptides and Sequences

Conserved sequences from various bacterial, viral, and parasitic genomes, and sequences containing multiple different epitopes are provided below which include sequences of interest that can be combined to form sequences. These sequences contain epitopes and/or mimotopes (listed below next to the sequence), that are recognized by mammalian immune systems. Epitopes are not sequences themselves, but specific structures in a region of an antigen or molecule that are recognized by immune system components such as lymphocytes (T and B cells), macrophages, dendritic cells, neutrophils, eosinophils, basophils, mast cells, and natural killer (NK) cells. An epitope is therefore determined by its structure and binding to a particular antibody. As such, there is variability to the sequence of amino acids as the structure determines the response, not the specific sequence. Individual amino acids of a sequence may be altered without altering the epitope structure and, in fact, individual amino acids do change from one organism to another without altering the fact that the sequence contains a specific epitope. Additional bacterial, viral, and other epitopes and antibodies and their corresponding paratopes are disclosed and discussed in U.S. Pat. Nos. 12,220,387; 12,076,390; 12,043,647; 12,023,384; 11,872,273; 11,866,463; 11,851,501; 11,640,847; 11,560,409; 11,439,702; 10,815,294; 10,774,134; 10,596,250; 10,004,799; 9,598,462; 9,777,045; 9,388,220; 8,821,885; and 8,470,340, and U.S Patent Application Publication Nos. 2024/0166697; 2024/0123055; 2024/0091331; 2024/0050552; 2023/0201326; and 2022/0280634, each of which is specifically incorporated by reference. The following is a list of such exemplary peptide sequences and the epitopes/mimotopes contained therein:

Influenza Virus

| | |
|---|---|
| SEQ ID NO 1 | DWSGYSGSFVQHPELTGLD (N1 sequence; H1 N5) |
| SEQ ID NO 2 | ETPIRNE (M2e epitope) |
| SEQ ID NO 3 | FVIREPFISCSHLEC |
| SEQ ID NO 4 | GNFIAP (HA epitope; Pep 1) |
| SEQ ID NO 5 | GNLIAP (HA epitope; Pep 2) |
| SEQ ID NO 6 | GNLFIAP (composite sequence of SEQ ID NOs 4 and 5; Pep 3) |
| SEQ ID NO 7 | GNLIFAP (composite sequence of SEQ ID NOs 4 and 5) |
| SEQ ID NO 8 | HYEECSCY (NA epitope; Pep 10) |
| SEQ ID NO 9 | LLTEVETPIR (highly conserved region M1/M2e) |
| SEQ ID NO 10 | LLTEVETPIRN (highly conserved region M1/M2e) |
| SEQ ID NO 11 | LLTEVETPIRNE (highly conserved region M1/M2e) |
| SEQ ID NO 12 | DWSGYSGSFVQHPELTGL (N1 sequence; H1 N5) |
| SEQ ID NO 13 | EVETPIRNE (highly conserved region M1/M2e) |
| SEQ ID NO 14 | FLLPEDETPIRNEWGLLTDDETPIRYIKANSKFIGITE |
| SEQ ID NO 15 | GNLFIAPGNLFIAPHYEECSCYHYEECSCYQYIKANSKFIGITEHY |
| | EECSCYTPIRNETPIRNE |
| SEQ ID NO 16 | GNLFIAPGNLFIAPQYIKANSKFIGITEGNLFIAP (composite of SEQ |
| | ID NO 6, SEQ ID NO 6, SEQ ID NO 61, and SEQ ID NO 6) |
| SEQ ID NO 17 | HYEECSCYDWSGYSGSFVQHPELTGLHYEECSCYQYIKAN |
| | SKFIGITE |
| SEQ ID NO 18 | ITGFAPFSKDNSIRLSAGGDIWVTREPYVSCDP |
| SEQ ID NO 19 | IWGIHHP (HA epitope) |
| SEQ ID NO 20 | IWGVHHP (HA epitope) |
| SEQ ID NO 21 | IWGVIHHP (composite of SEQ ID NOs. 19 and 20) |
| SEQ ID NO 22 | IWGIVHHP (composite of SEQ ID NOs. 19 and 20) |
| SEQ ID NO 23 | KSCINRCFYVELIRGR (N3 conserved epitope) |
| SEQ ID NO 24 | LLTEVETPIRNESLLTEVETPIRNEWG (M2e epitope) |
| SEQ ID NO 25 | LLTEVETPIRNEW (M2e epitope) |
| SEQ ID NO 26 | LLTEVETPIRNEWG (M2e epitope) |
| SEQ ID NO 27 | LTEVETPIRNE (M2e epitope) |
| SEQ ID NO 28 | LTEVETPIRNEW (M2e epitope) |
| SEQ ID NO 29 | LTEVETPIRNEWG (M2e epitope) |
| SEQ ID NO 30 | MSLLTEVET (M2e epitope) |
| SEQ ID NO 31 | MSLLTEVETP (M2e epitope) |
| SEQ ID NO 32 | MSLLTEVETPI (M2e epitope) |
| SEQ ID NO 33 | MSLLTEVETPIR (M2e epitope) |
| SEQ ID NO 34 | MSLLTEVETPIRN (M2e epitope) |
| SEQ ID NO 35 | MSLLTEVETPIRNE (M2e epitopes) |
| SEQ ID NO 36 | MSLLTEVETPIRNETPIRNE (M2e epitope) |
| SEQ ID NO 37 | MSLLTEVETPIRNEW (M2e epitope) |
| SEQ ID NO 38 | MSLLTEVETPIRNEWG (M2e epitope) |
| SEQ ID NO 39 | MSLLTEVETPIRNEWGCRCNDSSD (M2e epitope) |
| SEQ ID NO 40 | SLLTEVET (M2e epitope) |
| SEQ ID NO 41 | SLLTEVETPIR (M2e epitope) |
| SEQ ID NO 42 | SLLTEVETPIRNE (M2e epitope) |
| SEQ ID NO 43 | SLLTEVETPIRNEW (M2e epitope) |
| SEQ ID NO 44 | SLLTEVETPIRNEWG (M2e epitope) |
| SEQ ID NO 45 | SLLTEVETPIRNEWGTPIRNE (M2e epitope) |
| SEQ ID NO 46 | SLLTEVETPIRNEWGTPIRNETPIRNE (M2e epitope) |
| SEQ ID NO 47 | SLLTEVETPIRNEWGTPIRNETPIRNETPIRNE (M2e epitopes) |
| SEQ ID NO 48 | SLLTEVETPIRNEWGLLTEVETPIR (M1/M2e conserved region) |
| SEQ ID NO 49 | TEVETPIRNE (M2e epitope) |
| SEQ ID NO 50 | TPIRNE (M2e epitope) |
| SEQ ID NO 51 | VETPIRNE (M2e epitope) |
| SEQ ID NO 52 | VTREPYVSCDPKSCINRCFYVELIRGRVTREPYVSCDPWYIK |
| | ANSKFIGITE |
| SEQ ID NO 53 | WGIHHP (HA conserved region; Pep 5) |
| SEQ ID NO 54 | WGVHHP (HA conserved region; Pep 4) |
| SEQ ID NO 55 | WGVIHHP (composite of SEQ ID NOs 53 and 54; Pep 6) |
| SEQ ID NO 56 | WGIVHHP (composite of SEQ ID NOs 53 and 54; Pep 7) |
| SEQ ID NO 57 | YIWGIHHP (HA conserved region) |
| SEQ ID NO 58 | YIWGVHHP (HA conserved region) |
| SEQ ID NO 59 | YIWGVIHHP (composite of SEQ ID NOs 57 and 58) |
| SEQ ID NO 60 | YIWGIVHHP (composite of SEQ ID NOs 57 and 58) |
| SEQ ID NO 61 | QYIKANSKFIGITE (Tetanus T-cell epitope) |
| SEQ ID NO 62 | PIRNEWGCRCNDSSD (M2e epitope) |
| SEQ ID NO 63 | GNLFIAPWGVIHHPHYEECSCY (underlined sequences are epitopes |
| | HA {composite} (SEQ ID NO 6) and NA (SEQ ID NO 8), respectively, |
| | with middle as SEQ ID NO 55, of Influenza A; Pep 11) |
| SEQ ID NO 64 | CAGAGNFIAP |
| SEQ ID NO 65 | CAGAGNLIAP |
| SEQ ID NO 66 | CAGAGNLFIAP |
| SEQ ID NO 67 | CAGAWGVHHP |
| SEQ ID NO 68 | CAGAWGIHHP |
| SEQ ID NO 69 | CAGAWGVIHHP |
| SEQ ID NO 70 | CAGAWGIVHHP |
| SEQ ID NO 71 | GNLIAPWGVIHHP |
| SEQ ID NO 72 | CAGAGNLIAPWGVIHHP |
| SEQ ID NO 73 | GNLFIAPWGVIHHP |
| SEQ ID NO 74 | CAGAGNLFIAPWGVIHHP |
| SEQ ID NO 75 | CAGAHYEECSCY |
| SEQ ID NO 76 | CAGAGNLFIAPWGVIHHPHYEECSCY |
| SEQ ID NO 77 | GNLFIAPWGVIHHPGNLFIAPWGVIHHP |
| SEQ ID NO 78 | CAGAGNLFIAPWGVIHHPGNLFIAPWGVIHHP |
| SEQ ID NO 79 | HYEECSCYGNLFIAPWGVIHHP |
| SEQ ID NO 80 | GNLFIAPHYEECSCYWGVIHHP |
| SEQ ID NO 81 | SLLTEVETPIRNEWGLLTEVETPIRQYIKANSKFIGITE (Pep 5906; |
| | conserved matrix region (M1/M2e) plus T cell epitope) |
| SEQ ID NO 82 | VTREPYVSCDPKSCINRCFYVELIRGRVTREPYVSCDPQYIKANSKFIGITE |
| SEQ ID NO 83 | GNLFIAPRYAFA |
| SEQ ID NO 84 | CAGAGNLFIAPRYAFA |
| SEQ ID NO 85 | GNLVVPRYAFA |
| SEQ ID NO 86 | CAGAGNLVVPRYAFA |
| SEQ ID NO 87 | GNLIAPRYAFA |
| SEQ ID NO 88 | CAGAGNLIAPRYAFA |
| SEQ ID NO 89 | GNLVVP |
| SEQ ID NO 90 | CAGAGNLVVP |
| SEQ ID NO 91 | CAGAFVIREPFISCSHLEC |
| SEQ ID NO 92 | GNLFIAPWGVIHHPHYEECSCYQYIKANSKFIGITE |
| | (Pep 11 with C terminal T cell epitope = Pep 63) |
| SEQ ID NO 93 | QYIKANSKFIGITEGNLFIAPWGVIHHPHYEECSCY |
| | (Pep 11 with N terminal T cell epitope = Pep 64) |
| SEQ ID NO 94 | QYIKANSKFIGITEGNLFIAPWGVIHHPHYEECSCYTEVETPIRNE |
| | (Pep 11 with matrix epitope plus N terminal T cell epitope = Pep 64) |
| SEQ ID NO 95 | HVEECSY (N1 and N2) |
| SEQ ID NO 96 | WFIHHP (H5) |
| SEQ ID NO 97 | DLWSYNAELLV (stem peptide) |
| SEQ ID NO 98 | DIWTYNAELLV (stem peptide) |
| | HXXXW- matrix peptide common to Flu A and B that constitutes the |
| | main functional element of the M2 channel |

Coronavirus

| | |
|---|---|
| SEQ ID NO 99 | YPKCDRA = RNA Polymerase region |
| SEQ ID NO 100 | WDYPKCDRA = RNA Polymerase region (neutralizing Ab) |
| | Five coronavirus composite sequences using conserved epitopes. |
| SEQ ID NO 101 | SLDQINVTFLDLEYEMKKLEESY |
| | (coronavirus spike protein conserved epitope (SP)) |
| SEQ ID NO 102 | SLDQINVTFLDLEYEMKKLEESYQYIKANSKFIGITE |
| | (coronavirus spike protein conserved epitope (SP)) tetanus toxoid |
| | T cell epitope + SP) |
| SEQ ID NO 103 | WDYPKCDRAQYIKANSKFIGITE |
| | (POL + tetanus T cell epitope) |
| SEQ ID NO 104 | WDYPKCDRASLDQINVTFLDLEYEMKKLEESYQYIKANSKFIGITE |
| | (Cor POL + SP + Tet) |
| SEQ ID NO 105 | WDYPKCDRATEVETPIRNEHYEECSCYQYIKANSKFIGITE |
| | Cor POL. Flu M2e. Flu NA. Tetanus T cell |
| | (One coronavirus conserved epitope and two Flu conserved epitopes |
| | that is a broader pandemic vaccine) |

### Coronavirus Peptides and Composite Coronavirus/Influenza Peptides

| | |
|---|---|
| SEQ ID NO 106 | ARDLICAQ |
| | (highly conserved cor seq-spike attachment same in all three-Cor |
| | MERS SARS) |
| SEQ ID NO 107 | KWPWYIWLGFIAGL (highly conserved cor seq-spike attachment) |
| SEQ ID NO 108 | ENQKLIAN (highly conserved cor seq-spike attachment) |
| SEQ ID NO 109 | ARDLICAQKWPWYIWLGFIAGLENQKLIAN |
| | (combination of conserved seqs w/o T cell epitope) |
| SEQ ID NO 110 | ENQKLIANARDLICAQ |
| | (combination of conserved seqs w/o T cell epitope) |
| SEQ ID NO 111 | WDYPKCDRAENQKLIANARDLICAQ |
| | (combination of conserved seqs w/o T cell epitope) |
| SEQ ID NO 112 | WDYPKCDRAENQKLIANKWPWYIWLGFIAGL |
| | (combination of conserved seqs w/o T cell epitope) |
| SEQ ID NO 113 | ARDLICAQENQKLIANWDYPKCDRAQYIKANSKFIGITE |
| | (combinations of cor conserved seqs w/ T cell epitope) |
| SEQ ID NO 114 | KWPWYIWLGFIAGLWDYPKCDRAQYIKANSKFIGITEARDLIC |
| | AQENQKLIANWDYPKCDRAQYIKANSKFIGITE |
| | (combination of cor conserved seqs w/ T cell epitope) |
| SEQ ID NO 115 | ARDLICAQENQKLIANQYIKANSKFIGITE ARDLICAQENQKLIAN |
| | WDYPKCDRAQYIKANSKFIGITE (combination of cor conserved seqs w/ T cell epitope) |
| SEQ ID NO 116 | WDYPKCDRATEVETPIRNEHYEECSCYQYIKANSKFIGITE |
| | ARDLICAQENQKLIANWDYPKCDRAQYIKANSKFIGITE |
| | (combination of cor plus Influenza conserved seqs w/ T cell epitope; Just |
| | bold = Cor; Italicized = m2e; Underlined = Flu; Bold and underlined-T-cell |
| SEQ ID NO 117 | HYEECSCYWDYPKCDRAVETPIRNEQYIKANSKFIGITE |
| | (combination of cor plus Influenza conserved seqs w/ T cell epitope) |
| SEQ ID NO 118 | ENQKLIANTEVETPIRNEHYEECSCYQYIKANSKFIGITE |
| | (Conserved SARS epitopes, Flu Pep53 (M2), Flu Pep10, Tetanus T-cell epitope) |
| SEQ ID NO 119 | AEKAGGGGGAEKA (PGN epitope with pentaglycine bridge) |
| SEQ ID NO 120 | AEKAEKAGGGGGAEKAEKA (PGN epitope with pentaglycine bridge) |
| SEQ ID NO 121 | QYIKANSKFIGITEAEKAGGGGAEKA (PGN epitope with pentaglycine |
| | bridge and w/ T cell epitope). |
| SEQ ID NO 122 | AEKAGGGGGAEKAQYIKANSKFIGITE (PGN epitope with |
| | pentaglycine bridge and w/ T cell epitope). |
| SEQ ID NO 123 | AEKA (PGN epitope) |
| SEQ ID NO 124 | AEKAGGGGG (PGN epitope with pentaglycine bridge) |
| SEQ ID NO 125 | GGGGG (pentaglycine bridge) |
| SEQ ID NO 126 | SEFAYGSFVRTVSLPVGADE (Conserved MTB Alpha Crystallin HSP |
| | Epitope) |
| SEQ ID NO 127 | SEFAYGSFVRTVSLPVGADEGNLFIAPWGVIHHPHYEECSCY |
| | (Conserved MTB Alpha Crystallin HSP Epitope and 2 conserved influenza HA |
| | epitopes and 1 conserved NA Epitope) |
| SEQ ID NO 128 | HSFKWLDSPRLR (Conserved MTB Lipoarabinomanin Mimotope) |
| SEQ ID NO 129 | ISLTEWSMWYRH (Conserved MTB Lipoarabinomanin Mimotope) |
| SEQ ID NO 130 | WRMYFSHRHAHLRSP (LTA Epitope) |
| SEQ ID NO 131 | WHWRHRIPLQLAAGR (LTA Epitope) |
| SEQ ID NO 132 | GNLFIAPWGVIHHPHYEECSCY (composite influenza peptide |
| | comprising HA and NA epitopes) |
| SEQ ID NO 133 | SEFAYGSFMRSVTLPPGADE (M. smegmatis peptide sequence) |
| SEQ ID NO 134 | AEKAGGGGGAEKASEFAYGSFVRTVSLPVGADE (PGN epitopes and |
| | MTB 16.3HSP (CR) with and without a T cell epitope). |
| SEQ ID NO 135 | AEKAGGGGGAEKASEFAYGSFVRTVSLPVGADEQYIKANSKFIGITE |
| | (PGN epitope and MTB 16.3HSP (CR) with and without a T cell epitope). |
| SEQ ID NO 136 | SEFAYGSFVRTVSLPVGADEAEKAGGGGGAEKA (PGN epitope and |
| | MTB 16.3HSP (CR) with and without a T cell epitope). |
| SEQ ID NO 137 | AEKAGGGGGSEFAYGSFVRTVSLPVGADEGGGGGAEKA (PGN |
| | epitope and MTB 16.3HSP (CR) with and without a T cell epitope). |
| SEQ ID NO 138 | AEKAGGGGGSEFAYGSFVRTVSLPVGADEGGGGGAEKAQYIKANSKFIGITE |
| | (PGN epitope and MTB 16.3HSP (CR) with and without a T cell epitope). |
| SEQ ID NO 139 | WRMYFSHRHAHLRSPGGGGGAEKAGGGGGQYIKANSKFIGITE |
| | (PGN and LTA peptides with a T cell epitope). |
| SEQ ID NO 140 | WHWRHRIPLQLAGRAEKAGGGGGWRMYFSHRHAHLRSPQYIKANSKFIGITE |
| | (PGN and LTA peptides with a T cell epitope). |
| SEQ ID NO 141 | YFPLQSYGFQPTNGVGYQPYR (Coronavirus peptide without a T cell |
| | epitope). |
| SEQ ID NO 142 | YFPLQSYGFQPTNGVGYQPYRQYIKANSKFIGITE (Coronavirus |
| | peptide with a T cell epitope). |
| SEQ ID NO 143 | YQAGSTPCNGVEGFNCYFPLQYIKANSKFIGITE (Coronavirus |
| | peptide with a T cell epitope). |
| SEQ ID NO 144 | YQAGSTPCNGVEGFNCYFPLQ (Coronavirus peptide without a T cell |
| | epitope). |
| SEQ ID NO 145 | NPDPNANPNVDPNANGGGG |
| | CSP Junctional Region Epitope-Induced Antibodies inhibit parasitic |
| | liver invasion Malaria epitope |
| SEQ ID NO 146 | RKSIHLGPGRAFY (HIV1) UG1033 |
| SEQ ID NO 147 | KKGIAIGPGRTLY (HIV2) NY5 |
| SEQ ID NO 148 | RKSIRIGPGQAFY (HIV3) ZAM18 |
| SEQ ID NO 149 | RKRIRVGPGQTVY (HIV4) NDF |
| | HIV Peptides: GP120 V3 Crown Variable Region Conserved Peptides |
| | (These crown peptides are targeted by cross-clade neutralizing Mabs) |
| SEQ ID NO 150 | CATGIAVAG (N-terminal domain epitope without T cell epitope). |
| SEQ ID NO 151 | YYYYYGMDVW (N-terminal domain epitope without T cell epitope). |
| SEQ ID NO 152 | CATGYSSSWYFDYW (N-terminal domain epitope without T cell |
| | epitope). |
| SEQ ID NO 153 | CAKGYSYGYNWFDSW (N-terminal domain epitope without T cell |
| | epitope). |
| SEQ ID NO 154 | CQQYNNWPPLTF (N-terminal domain epitope without T cell epitope). |
| SEQ ID NO 155 | CATGIAVAGQYIKANSKFIGITE (N-terminal domain epitope with T |
| | cell epitope). |
| SEQ ID NO 156 | YYYYYGMDVWQYIKANSKFIGITE (N-terminal domain epitope with |
| | T cell epitope). |
| SEQ ID NO 157 | CATGYSSSWYFDYWQYIKANSKFIGITE (N-terminal domain epitope |
| | with T cell epitope). |
| SEQ ID NO 158 | CAKGYSYGYNWFDSWQYIKANSKFIGITE (N-terminal domain |
| | epitope with T cell epitope). |
| SEQ ID NO 159 | CQQYNNWPPLTFQYIKANSKFIGITE (N-terminal domain epitope with |
| | T cell epitope). |
| SEQ ID NO 160 | ARDLICAQCATGYSSSWYFDYWQYIKANSKFIGITE (N-terminal |
| | domain epitope with T cell epitope). |
| SEQ ID NO 161 | WDYPKCDRATEVETPIRNEHYEECSCYCQQYNNWPPLTF |
| | QYIKANSKFIGITE (Conserved regions from the RNA polymerase epitope, |
| | Influenza peptide 52, Influenza peptide 10 (NA), N-terminal domain |
| | epitope with T cell epitope). |
| SEQ ID NO 162 | WDYPKCDRATEVETPIRNEARDLICAQENQKLIANCATGIAVAG |
| | QYIKANSKDIGITE (Conserved regions from the RNA polymerase epitope, |
| | Influenza peptide 52, N-terminal domain epitope with T cell epitope). |
| SEQ ID NO 163 | WDYPKCDRAENQKLIANARDLICAQCATGYSSSWYFDYW |
| | QYIKANSKFIGITE (Conserved regions from the RNA polymerase epitope, |
| | N-terminal domain epitope with T cell epitope). |

### Example 2 Induction of Neutralizing Antibodies with Peptides

ICR mice and cotton rats were immunized with 1 μg of conjugated or unconjugated influenza peptide vaccine (influenza HA, NA and M2e peptides with a T-cell epitope) formulated with ALFQ by intramuscular, or intradermal routes (cotton rats were given both intramuscular, or intradermal injections). Both routes of administration induced serum IgG antibodies that bound to groups 1 and 2 influenza viruses (Flu A California H1N1/pdm09 and Flu A Hong Kong H3N2/4801/2014). In addition, 1 μg of influenza vaccine formulated in ALFQ induced neutralizing antibodies against both influenza viruses given by intradermal and intramuscular routes. These data demonstrate that influenza peptide vaccines formulated in ALFQ induced a strong immune response at a very low dose without conjugation to a carrier and when administered by different routes of immunization. This provides an advantage in efficiency of manufacturing and decreased cost of production. Low dose intradermal administration also decreases vaccine costs for mass global immunization of humans and for immunizing mammals such as humans or animals such as birds and pigs.

### Example 3 Universal Influenza Vaccine

The influenza peptide vaccine induced serum antibodies that bind broadly across groups 1 and 2 influenza A viruses (to include pandemic and avian influenza strains) and influenza B virus. The influenza peptide vaccine induced serum antibodies that neutralize influenza A viruses. Anti-Flu Pep 6 MAB LD9 targets Hemagglutinin (HA) and binds to pandemic and avian influenza strains and influenza B virus. Anti-Flu Pep 10 MAB NB5 targets Neuraminidase (NA) and binds to pandemic and avian influenza strains and influenza B virus. Anti-Flu Pep 5906 MAB GA4 targets Matrix Ectodomain (M2e), binds to pandemic and avian influenza strains and influenza B virus. Neutralizing MABs LD9, NB5, and GA4 that bind to these influenza peptide epitopes (HA, NA, and M2e, respectively) target both seasonal influenza A strains and pandemic/avian influenza strains to include H5N1 and H5N6. In addition, the neutralizing MABs to these peptides also bind strongly to influenza B.

### Example 4 Peptide TB, Gram Positive Bacteria and Influenza/Coronavirus Vaccines

The 16.3 KD alpha crystallin heat shock protein (HSP16.3) belongs to the small heat shock protein (HSP20) family. It plays a major role for MTB survival, growth, virulence and cell wall thickening. TB Pep 01 is a highly conserved region of HSP16.3 and immunization of mice induced antibodies that bind to mycobacteria and promote opsonophagocytic killing of M. smegmatis. Peptidoglycan is a cell wall component that is common across many bacteria and antibodies to PGN bind to MTB (and other gram-positive bacteria). Immunization of mice with ethanol killed MTB induced anti-PGN antibodies that promoted phagocytic killing of MTB. In addition, these antibodies bind to small PGN epitopes (Table 2) and antigens (SEQ ID NO 119-138). Cell wall PGN (SEQ ID NO 119) peptides and HSP16.3 (SEQ ID NO 126) the highly conserved peptide (TB Pep 01) can be mixed and matched to produce peptides and mixtures with or without an added T cell epitope to provide vaccines to produce broadly protective immunity across large groups of bacteria.

TABLE 2
PGN Peptide Sequences

| SEQ | Peptide | | |
|---|---|---|---|
| ID NO | number | Peptide ID | Peptide Sequence |
| 119 | PGN Pep01 | LVD-PSEQ-A- | AEKAGGGGGAEKA |
| | | PGN Pep 01 | |
| 120 | PGN Pep02 | LVD-PSEQ-A- | AEKAEKAGGGGGAEKA |
| | | PGN Pep 02 | EKA |
| 121 | PGN Pep03 | LVD-PSEQ-A- | QYIKANSKFIGITEAE |
| | | PGN Pep 03 | KAGGGGAEKA |
| 122 | PGN Pep04 | LVD-PSEQ-A- | AEKAGGGGGAEKAQYI |
| | | PGN Pep 04 | KANSKFIGITE |
| 123 | PGN Pep05 | LVD-PSEQ-A- | AEKA |
| | | PGN Pep 05 | |
| 124 | PGN Pep06 | LVD-PSEQ-A- | AEKAGGGGG |
| | | PGN Pep 06 | |

In addition, combining HSP16.3 with PGN epitopes provides a TB vaccine that targets active MTB infection and latency. This vaccine could be used alone, or in combination with BCG and could be used as a booster vaccine with BCG, or other TB vaccines. In a similar fashion, LTA mimotopes combined with PGN epitopes (Table 3) provide an example of a broad peptide gram positive bacterial vaccine, while mixing coronavirus and influenza peptides provides a prototype peptide vaccine for prevention or treatment of infections by these viruses.

TABLE 3
MTB, PGN, and LTA Peptide Sequences

| SEQ | Peptide | | |
|---|---|---|---|
| ID NO | number | Peptide ID | Peptide Sequence |
| 126 | TB | LVD-PSEQ- | SEFAYGSFVRTVSLPV |
| | Pep01 | A-TB | GADE |
| | | Pep01 | |
| 134 | PGN.TB | LVD-PSEQ- | AEKAGGGGGAEKASEF |
| | Pep01 | A-PGN.TB | AYGSFVRTVSLPVGAD |
| | | Pep01 | E |
| 135 | PGN.TB | LVD-PSEQ- | AEKAGGGGGAEKASEF |
| | Pep02 | A-PGN.TB | AYGSFVRTVSLPVGAD |
| | | Pep02 | EQYIKANSKFIGITE |
| 136 | PGN.TB | LVD-PSEQ- | SEFAYGSFVRTVSLPV |
| | Pep03 | A-PGN.TB | GADEAEKAGGGGGAEK |
| | | Pep03 | A |
| 137 | PGN.TB | LVD-PSEQ- | AEKAGGGGGSEFAYGS |
| | Pep04 | A-PGN.TB | FVRTVSLPVGADEGGG |
| | | Pep04 | GGAEKA |
| 138 | PGN.TB | LVD-PSEQ- | AEKAGGGGGSEFAYGS |
| | Pep05 | A-PGN.TB | FVRTVSLPVGADEGGG |
| | | Pep05 | GGAEKAQYIKANSKFI |
| | | | GITE |
| 139 | PGN.LTA | LVD-PSEQ- | WRMYFSHRHAHLRSPG |
| | Pep01 | A- | GGGGAEKAGGGGGQYI |
| | | PGN.LTA | KANSKFIGITE |
| | | Pep01 | |
| 140 | PGN.LTA | LVD-PSEQ- | WHWRHRIPLQLAGRAE |
| | Pep02 | A- | KAGGGGGWRMYFSHRH |
| | | PGN.LTA | AHLRSPQYIKANSKFI |
| | | Pep02 | GITE |

Studies in ICR mice have also demonstrated that immunization with unconjugated TB Pep 1 plus PGN formylated with ADDAVAX™ adjuvant induced robust serum antibody responses with doses as low as 10-20 μg of each peptide/antigen. Antibodies broadly targeted bacteria to include Mycobacteria, Staphylococci, Streptococci, and Bacillus species. In addition, antibodies were shown to promote opsonophagocytic killing of bacteria by U937 macrophages. This HSP16.3 and PGN vaccine that covers multiple pathogens provides a cost effective and easily scalable approached for a vaccine to target TB and gram-positive pathogens.

### Example 5 MABs to PGN are Opsonic

MABs JG7 and GG9 showed binding activity to killed MTB, live Mycobacterium smegmatis (SMEG) and several strains of live MTB-susceptible, MDR and XDR. In addition, JG7 and GG9 promoted opsonophagocytic killing of SMEG and MTB using macrophage and granulocytic cell lines and enhanced clearance of MTB from blood.

Monoclonal antibodies (MABs) JG7, GG9, and MD11 were developed against a Mycobacterium tuberculosis (MTB) and gram-positive bacteria cell wall component peptidoglycan (PGN). Mouse splenocytes were fused with SP2/0 myeloma cells for production of hybridomas and MABs. MAB JG7 (IgG1) was derived from BALB/c MS 1323 immunized intravenously with Ethanol-killed Mycobacterium tuberculosis (EK-MTB), without adjuvant. Killing of MTB using Ethanol may have altered the MTB capsule exposing deeper cell wall epitopes. MAB GG9 (IgG1) was derived from BALB/c MS 1420 immunized subcutaneously with EK-MTB, without adjuvant. MAB MD11 (IgG2b) was derived from ICR MS 190 immunized subcutaneously with ultrapure Peptidoglycan (PGN), conjugated to CRM197 and adjuvanted with TiterMax® Gold. EK-MTB and PGN were immunogenic in mice. Serum antibodies that bound to gram-positive bacteria and MTB and promoted opsonophagocytic killing (OPKA) of the bacteria by phagocytic effector cells.

Binding activities of supernatants from hybridomas JG7 and GG9 (from mice 1323 and 1420, respectively), to Mycobacterium tuberculosis (MTB) and Mycobacterium smegmatis (SMEG), evaluated at dilutions 1:10, 1:100, and 1:1000 on fixed mycobacteria at 1×10⁵ CFU/well. Binding of supernatant to killed MTB Erdman, HN878 and CDC1551. Binding of supernatants to fixed SMEG. OD values for growth media without antibody (negative control) range between 0.046-0.060. Binding activity of purified anti-Mycobacterium tuberculosis monoclonal antibodies (anti-MTB MABs) GG9 and JG7 to live Mycobacterium smegmatis (SMEG) and live susceptible MTB H37Ra (lab strain) and STB1 and STB2 (susceptible clinical isolates) was demonstrated in a live bacteria ELISA. Data are representative of three individual experiments.

Demonstrated binding activity of purified anti-Mycobacterium tuberculosis monoclonal antibodies (anti-MTB MABs) JG7 and GG9 to fixed MTB at 1×10⁵ CFU/well. Demonstrated MAB binding to susceptible H37Ra strain and clinical isolates 1, and 2; to multidrug-resistant (MDR) clinical isolates 1, 2 & 3; and to extensively drug-resistant (XDR) clinical isolates 1 and 2. Data (expressed as mean) are representative of three individual experiments. Demonstrated binding activity of anti-MTB MABs JG7 & GG9 to various live gram-positive bacteria grown to either log phase or stationary phase as screened in the live bacteria ELISA. Enhanced OPKA of MABS JG7 and GG9 against Mycobacterium smegmatis (SMEG) using HL60 granulocytes and C1q occurred at low antibody concentrations (<0.25 μg/ml) and stayed constant when antibody levels were increased over one hundred-fold. While MAB JG7 consistently had higher percent killing, the difference did not reach statistical significance. Peak OPKA for both JG7 and GG9 occurred at 0.06 μg/mL and were 81% and 76%, respectively. Enhanced MAB OPKA against SMEG using U-937 macrophages (without C1q) was significantly more pronounced at higher antibody concentrations (JG7: p=0.0001, GG9: p<0.0001) and both MABs tracked closely together across all antibody concentrations. Peak OPKA for JG7 and GG9 were 82% at 175 μg/mL and 76% at 100 μg/mL, respectively.

To summarize, these monoclonal antibodies bound to multiple MTB strains, M. smegmatis, and susceptible, MDR, and XDR clinical isolates.

OPKA of MAB JG7 against live Mycobacterium tuberculosis (MTB) clinical isolate STB1, using U-937 macrophages (without C1q) was significantly enhanced at MAB levels 2.5-25 μg/mL. Compared to the control sample wells (without MAB), antibody sample wells had CFU counts that were significantly reduced (p<0.5) from 315 (No MAB) to 219 (2.5 μg/mL), 154 (5 μg/mL), 145 (10 μg/mL.) and 143 (25 μg/mL). Data (expressed as mean±standard errors; n=3) are representative of three individual experiments. The MABs also demonstrated broad bacterial binding and enhanced OPKA against MTB and M. smegmatis.

Using qPCR, rapid clearance of Mycobacterium tuberculosis (MTB) in blood was observed in all groups from the in vivo study with N=76 ICR mice. While MAB GG9 significantly enhanced blood clearance at 24 hours post challenge (1 mg/kg p=0.0021, 10 mg/kg p=0.0013), MAB JG7 significantly enhanced clearance at all time points (0.25, 4 and 24 hours) and at one or more doses. The percentage of mice with undetectable levels of MTB in blood according to qPCR. Statistical significance determined by comparison of MAB-treated vs. PBS-treated blood samples from mice according to no detection (i e., CT=40, qPCR) was calculated using the Chi-squared test, with significance threshold set at p<0.05 and 95% confidence intervals shown. In addition, the MABs promoted rapid clearance of MTB from the blood of mice given as little as 1 mg/kg.

MABs JG7 and GG9 and anti-LTA MAB (96-110) were analyzed for binding to a cell wall mixture and Ultrapure PGN, both from Staphylococcus aureus. Compared to a control MAB 96-110 directed against LTA that only bound to impure cell wall mixture containing components including LTA and PGN, MABs JG7 and GG9 bound to both cell wall mixture and ultrapure PGN (that does not contain other cell wall components such as LTA). This strongly suggests that MABs JG7 and GG9 bind to an epitope on PGN. PGN-binding activity of MABs GG9 and JG7 was demonstrated to Ultrapure and Impure PGN, while anti-LTA MAB 96-110 only bound the Impure PGN. MABs JG7 and GG9 are IgG1 and both MABs bound to ultra-pure peptidoglycan (PGN).

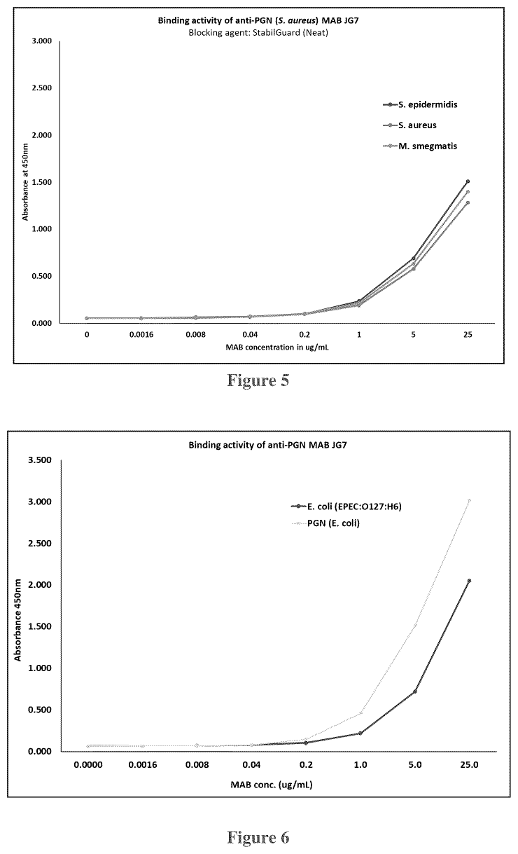

As shown in FIG. 5, MAB JG7 was analyzed for binding activity to gram-positive bacteria including mycobacteria. At concentrations between 1-25 μg/mL, MAB JG7 bound well to Staphylococcus aureus and Staphylococcus epidermidis strains, and also bound to Mycobacterium smegmatis (SMEG). Opsonophagocytic killing activity of MAB JG7 against S. epidermidis was demonstrated (46%) using macrophage cell line U-937s.

As shown in FIG. 6, MAB JG7 was analyzed for binding activity to ultra-pure PGN derived from Escherichia coli (E. coli) and to gram-negative E. coli bacteria. At concentrations between 1-25 μg/mL, MAB JG7 bound well to PGN (E. coli derived) and to whole E. coli (EPEC:0127:H6 strain). Opsonophagocytic killing activity of MAB JG7 against E. coli was demonstrated (49%) using macrophage cell line U-937s.

Mice were subsequently immunized with CRM-conjugated PGN, and serum antibodies were induced that also reacted broadly across gram-positive bacteria and MTB. Moreover, the mice produced serum antibodies that bound to PGN and fixed bacteria. Mouse 190 (MS 190) serum antibodies showed good binding to ultrapure PGN (d42), bound broadly to various gram-positive bacteria, and enhanced OPKA of M. smegmatis. Mouse 190 (MS 190) with anti-PGN serum antibodies that also bound broadly to bacteria and enhanced OPKA was selected for hybridoma production.

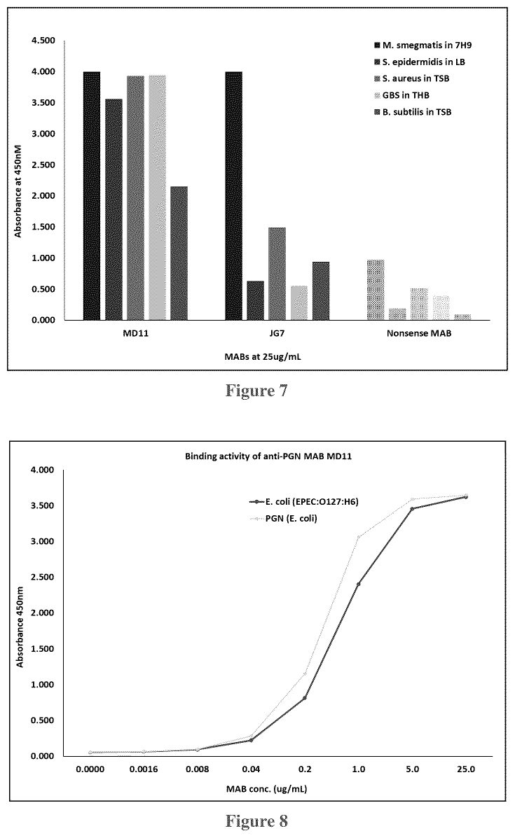

MS190 Hybridoma clone MD11 is an IgG2b MAB that binds across multiple bacteria and ultra-pure PGN which was identified from the hybridomas that were produced. MAB MD11 showed binding activity to Peptidoglycan, killed MTB, and various strains of gram-positive bacteria and recognized M. smegmatis (SMEG), S. epidermidis, S. aureus, Group B Streptococcus (GBS), and Bacillus subtilis (FIG. 7). Conjugated PGN immunization induced broadly reactive antibodies to bacteria Similarly, MAB JG7 (at 25 μg/mL) showed strong binding activity to M. smegmatis (SMEG); however, compared with MD11, JG7 binding was lower with S. epidermidis, S. aureus, Group B Streptococcus (GBS), and Bacillus subtilis (FIG. 7).

Competitive inhibition of MAB MD11 binding to ultrapure PGN by small synthetic PGN peptides. Competitive inhibition of MAB MD11 binding to the small synthetic PGN peptides by Ultrapure PGN derived from S. aureus. In addition, MD11 bound to SMEG and promoted opsonophagocytic killing of SMEG and Staphylococci (>50% OPKA) using macrophages (U-937 cell line) and polymorphonuclear cells (PMNs; HL60 granulocytes), respectively.

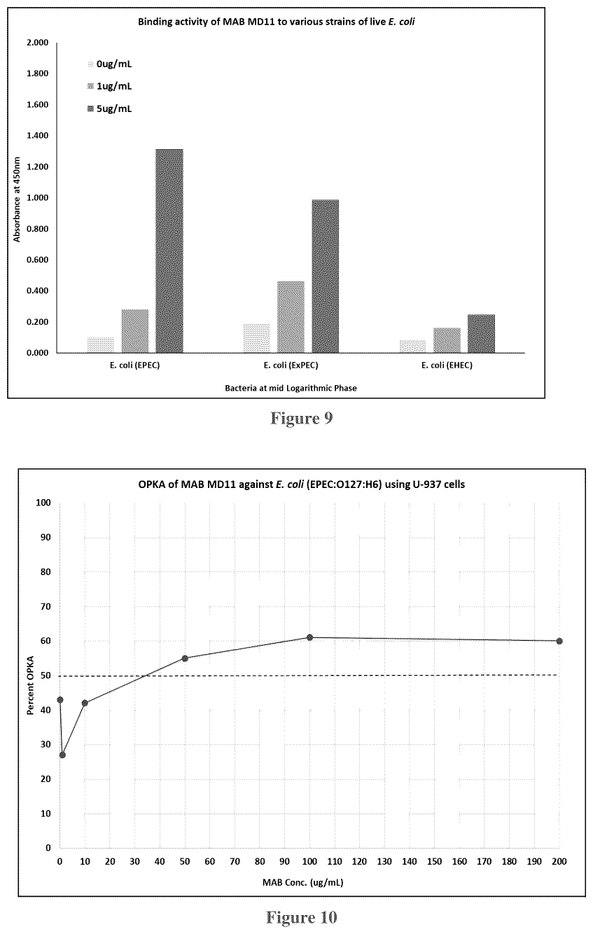

MAB MD11 was analyzed for binding activity to ultra-pure PGN derived from E. coli and to gram-negative E. coli bacteria. At concentrations between 0 05-25 μg/mL, MAB MD11 bound well to PGN (E. coli derived) and to whole E. coli (EPEC:0127:H6 strain) (FIG. 8). At concentrations greater than 1 μg/mL, MAB MD11 bound to various strains of live gram-negative bacteria (E. coli-EPEC:0127:H6, ExPEC:017:K52:H18, and EHEC:0157:H7) (FIG. 9). Opsonophagocytic killing activity of MAB MD11 against E. coli (EPEC:0127:H6 strain) was demonstrated with significance (>50%) using macrophage cell line U-937s (FIG. 10). MAB MD11 was also analyzed for binding activity to live Mycobacterium tuberculosis (MTB) at mid-logarithmic and stationary phases of MTB growth. MD11 demonstrated good binding activity to live MTB at mid-logarithmic and stationary phases of MTB growth (FIG. 10A) and also bound strongly to ethanol-killed MTB that was also grown at mid-Log and stationary phases of growth, and showed a dose response with increasing concentration of antibodies (FIG. 10B).

### Example 6 Peptidoglycan Peptides and Antigens

MABs JG7, GG9 and MD11 were analyzed for binding to small, synthesized peptides and to ultra-pure PGN. MABs JG7 and GG9 are from mice immunized with ethanol killed MTB and MAB MD11 from a mouse immunized with CRM-conjugated PGN. Each of the MABs bound to all the small individual peptides (Table 1) and to PGN, but the binding patterns across the peptides were different.

### Example 7 Monoclonal Antibodies to Peptides

Monoclonal antibodies (MABs) were developed against Mycobacterium tuberculosis Alpha Crystallin Heat Shock Protein. MAB LD7 (IgG2a) was derived from BALB/c MS 1435 immunized subcutaneously with TB Pep01 (Conserved Alpha Crystallin HSP), with Freund's adjuvant. MAB CA6 (IgG2b) was derived from BALB/c MS 1435 immunized subcutaneously with TB Pep01 (Conserved Alpha Crystallin HSP), with Freund's adjuvant.

PGN epitopes shown in Table 1 can be mixed and matched in varied combinations such as with or without a T cell epitope, to produce peptides and mixtures that could be formulated with adjuvants as MTB or Staph/Gram positive bacterial vaccines.

TABLE 4
MTB, LAM, and Staphylococcus LTA Peptide Sequences

| SEQ | | | | |
|---|---|---|---|---|
| ID | Peptide | Peptide | Peptide | |
| NO | number | ID | Sequence | Description |
| 126 | TB | LVD-PSEQ- | SEFAYGSF | Conserved MTB |
| | Pep01 | A-TB | VRTVSLPV | Alpha Crystallin |
| | | Pep 01 | GADE | HSP Epitope |
| 127 | TB | LVD-PSEQ- | SEFAYGSF | Conserved MTB |
| | Pep02 | A-TB | VRTVSLPV | Alpha Crystallin |
| | | Pep 02 | GADEGNLF | HSP Epitope and |
| | | | IAPWGVIH | 2 conserved |
| | | | HPHYEECS | influenza HA |
| | | | CY | epitopes and 1 |
| | | | | conserved NA |
| | | | | Epitope |
| 128 | LAM | LVD-PSEQ- | HSFKWLDS | Conserved MTB |
| | Pep01 | A-LAM | PRLR | Lipoarabinomanin |
| | | Pep 01 | | Mimetope |
| 129 | LAM | LVD-PSEQ- | ISLTEWSM | Conserved MTB |
| | Pep02 | A-LAM | WYRH | Lipoarabinomanin |
| | | Pep 02 | | Mimetope |
| 130 | LTA | LVD-PSEQ- | WRMYFSHR | LTA Epitope |
| | Pep01 | A-LTA | HAHLRSP | |
| | | Pep 01 | | |
| 131 | LTA | LVD-PSEQ- | WHWRHRIP | LTA Epitope |
| | Pep02 | A-LTA | LQLAAGR | |
| | | Pep 02 | | |

MTB, LAM and Staphylococcus LTA epitopes shown in Table 4 can be mixed and matched in combinations such as with or without a T cell epitope, to produce peptides and mixtures that could be formulated with adjuvants as MTB or Staph/Gram positive bacterial vaccines.

Binding activities of supernatants from hybridomas LD7 and CA6 to TB Pep01 and TB Pep02 at 1 μg/mL, to live Mycobacterium smegmatis (SMEG) in mid-log phase, live, heat-treated or ethanol-treated SMEG, and to live Mycobacterium smegmatis (SMEG) as demonstrated in a Live Bacteria ELISA. MABs LD7 and CA6 showed highly specific binding to the alpha crystallin HSP (TB Pep01) and promoted opsonophagocytic killing of M. smegmatis (SMEG). Enhanced OPKA of MABs LD7 and CA6 against Mycobacterium smegmatis (SMEG) using U-937 macrophages. Peak OPKA for LD7 was 76% and for CA6 was 63%. MABs were purified from hybridoma subclones and OD values (450 nM) for growth media without antibody (negative control) range between 0.046-0.060.

There is 80% homology (16 out of 20 amino acids) of HSP20 between M. tuberculosis (SEQ ID NO 126; SEFAYGSFVRTVSLPVGADE) and M. smegmatis (SEQ ID NO 133; SEFAYGSFMRSVTLPPGADE).

Mouse 1435 immunized with a conserved MTB alpha crystallin heat shock protein epitope developed serum antibodies that bound to a small synthesized alpha crystallin HSP peptide (TB Pep01). MAB LD7 (IgG2a) and MAB CA6 (IgG2b) that were subsequently produced from MS 1435 bound broadly to TB Pep01, TB Pep02 (peptide that constitutes TB Pep01, two conserved influenza hemagglutinin epitopes, and one conserved neuraminidase epitope), and M. smegmatis. In addition, these MABs showed enhanced OPKA (>50%) against M. smegmatis

The HSP epitope elicited strong humoral responses in mice, with high serum antibody titers and subsequently generated two MABs-LD7 and CA6 (IgG2a and IgG2b isotypes, respectively). These MABs bound strongly to the HSP epitope (OD450 nm of 3.0-3.5) but had low binding activity to fixed mycobacteria (OD450 nm<0.25). Notably, MABs LD7 and CA6 showed significantly increased binding activity to live SMEG, compared to fixed SMEG, and demonstrated significant OPKA against SMEG at both low (0.1 μg/mL) and high (200 μg/mL) antibody concentrations.

The small conserved synthetic HSP epitope induced a robust humoral response in mice and generated two MABs that recognized live SMEG and demonstrated significant OPKA against SMEG at MAB concentrations as low as 0.1 μg/mL. Immunization with this small conserved synthetic HSP epitope generates opsonic antibody responses against mycobacteria and provide important strategies for TB vaccines and therapeutics.

### Example 8 Peptide Vaccines for Influenza and Other Viruses

An influenza vaccine comprising small conserved epitopes such as HA, NA, or matrix peptide sequences induce broadly neutralizing antibodies across Group 1 and 2 Influenza A viruses. Combining one or more of these peptides with one or more small, conserved peptide sequences from two or more viruses (such as influenza and coronavirus) provides a prototype virus peptide vaccine that broadens the vaccine's prevention or treatment capabilities to include more than one virus. Combined influenza and coronavirus peptide vaccine antigens were synthesized and included the conserved influenza matrix and NA peptides plus the conserved coronavirus polymerase peptide (Cor Pep 05), or spike protein conserved sequence (Cor Pep 11) and a T cell epitope sequence (Table 5). The polymerase conserved epitope was also sequenced alone with the T cell epitope (Cor Pep 02).

TABLE 5
Peptide Antigens for Influenza and Other Viruses

| SEQ | | | |
|---|---|---|---|
| ID | Peptide | | |
| NO | number | Peptide ID | Peptide Sequence |
| 6 | Flu Pep03 | LVD-PSEQ-A- | GNLFIAP |
| | | Flu Pep03 | |
| 55 | Flu Pep06 | LVD-PSEQ-A- | WGVIHHP |
| | | Flu Pep06 | |
| 8 | Flu Pep10 | LVD-PSEQ-A- | HYEECSCY |
| | | Flu Pep10 | |
| 141 | Cor Pep13 | LVD-PSEQ-A- | YFPLQSYGFQPTNGV |
| | | Coronavirus | GYQPYR |
| | | Pep13 | |
| 142 | Cor Pep14 | LVD-PSEQ-A- | YFPLQSYGFQPTNGV |
| | | Coronavirus | GYQPYRQYIKANSKF |
| | | Pep14 | IGITE |
| 144 | Cor Pep15 | LVD-PSEQ-A- | YQAGSTPCNGVEGFN |
| | | Coronavirus | CYFPLQ |
| | | Pep15 | |
| 143 | Cor Pep16 | LVD-PSEQ-A- | YQAGSTPCNGVEGFN |
| | | Coronavirus | CYFPLQYIKANSKFI |
| | | Pep16 | GITE |
| 2 | Flu Pep52 | LVD-PSEQ-A- | ETPIRNE |
| | | Flu Pep52 | |
| 49 | Flu Pep53 | LVD-PSEQ-A- | TEVETPIRNE |
| | | Flu Pep53 | |
| 48 | Flu Pep57 | LVD-PSEQ-A- | SLLTEVETPIRNEWG |
| | | Flu Pep57 | LLTEVETPIR |
| 100 | Cor Pep01 | LVD-PSEQ-A- | WDYPKCDRA |
| | | Cor Pep 01 | |
| 103 | Cor Pep02 | LVD-PSEQ-A- | WDYPKCDRAQYIKAN |
| | | Cor Pep 02 | SKFIGITE |
| 105 | Cor Pep05 | LVD-PSEQ-A- | WDYPKCDRATEVETP |
| | | Cor Pep 05 | IRNEHYEECSCYQYI |
| | | | KANSKFIGITE |
| 108 | Cor Pep09 | LVD-PSEQ-A- | ENQKLIAN |
| | | Cor Pep 09 | |
| 118 | Cor Pep11 | LVD-PSEQ-A- | ENQKLIANTEVETPI |
| | | Cor Pep 11 | RNEHYEECSCYQYIK |
| | | | ANSKFIGITE |

Mice were immunized with one, or more of these peptides formulated with ADDAVAX™ adjuvant and given by either subcutaneous (SQ) injection at a dose of 20 μg, or Intradermal (ID) injection at 1, 10, or 20 μg on days 0, 21 and 35. Robust serum IgG1 and IgG2b antibodies were induced to the conserved influenza and coronavirus epitopes and to whole coronavirus and influenza viruses. Serum antibody responses in mice immunized subcutaneously with 20 μg dose of Coronavirus Pep02, Coronavirus Pep05, or Coronavirus Pep11 and booster immunizations given on Days 21 and 35. Profile of IgG1 antisera titers or IgG2b antisera titers to the immunogens are shown as Mean±SD. Serum antibody responses in mice immunized with Coronavirus Pep02 and Coronavirus Pep05. IgG1 antisera titers to the coronavirus peptides are shown in IgG2b titers to the same peptides. Serum antibody responses in mice immunized with Coronavirus Pep05 and Coronavirus Pep11. Profile of IgG1 or IgG2b antisera titers to the coronavirus peptides; titers to influenza epitopes and titers to individual coronavirus RNA polymerase and spike protein epitopes.

In addition, the antisera titers rose rapidly to the polymerase and spike coronavirus epitopes on the homologous peptide antigens and to the influenza epitopes on the antigens. The peptide antigens that included both coronavirus and influenza peptides with a T-cell epitope, provided a greater response than the peptide with the coronavirus polymerase epitope and a T-cell epitope. In addition, comparing the IgG responses to polymerase and spike protein epitopes showed dramatically different profiles with antibodies to polymerase steadily increasing over 49 days, while spike antibodies went up rapidly and either flattened or dropped between days 28 and 49. Antisera titers to the influenza epitopes increased rapidly and then leveled off after day 21.

Serum antibody responses in select mice immunized subcutaneously with 20 μg dose of either Coronavirus Pep11 or Coronavirus Pep05. One year post primary immunizations, the selected mice were given a boost and bled a week after. IgG1 antibody titers to coronavirus peptides and influenza epitopes. Serum antibody responses in select mice immunized subcutaneously with 20 μg dose of either Coronavirus Pep11 or Coronavirus Pep05. One year post primary immunizations, the selected mice were given a boost and bled a week after. IgG antibody titers to influenza virus A and IgG responses to human Coronavirus. Neutralizing titers in select mice immunized subcutaneously with 20 μg dose of either Coronavirus Pep11 or Coronavirus Pep05. One year post primary immunizations, the selected mice were given a boost and bled a week after. Neutralization of influenza A/Hong Kong (H3N2) (ID₇₅ values). Furthermore, the durability of the antibody responses were strong for both coronavirus and influenza peptides in the peptide antigens and for the antisera binding to influenza and coronavirus viruses one year after primary immunization.

Also, 70 days after initial immunization, antisera bound across Groups 1 (H1N1) and 2 (H3N2) influenza A viruses and influenza B virus with strong neutralization. IgG1 antisera titers (day 266) to human Coronavirus (hCoV) NL-63. End-point neutralization titers based on 75% neutralization of hCoV NL-63 are shown as PRNT75 values.

In addition, day 252 IgG1 antisera bound strongly across 3 variants of gamma-irradiated SARSCoV-2 variants re shown in mice immunized with Coronavirus Pep02, Coronavirus Pep05 and Coronavirus Pep11. Serum antibody responses were measured in mice immunized with a combination of Coronavirus Pep05 and Coronavirus Pep11. IgG1 and IgG2b antisera titers to the coronavirus peptides and influenza epitopes. Virus binding titers (IgG1) to various subtypes of influenza A and B and three variants of SARS-CoV-2.

Serum antibody responses in mice immunized intradermally with lug, 10 μg or 20 μg dose of a vaccine comprising of Coronavirus Pep11 and Coronavirus Pep05 and booster immunizations given on days 21 and 35. IgG1 antisera titers to the coronavirus peptides for each dose group, titers to influenza epitopes and universal T cell epitopes for each dose group.

Serum antibody responses in mice immunized intradermally with lug, 10 μg, or 20 μg dose of a vaccine comprising of Coronavirus Pep11 and Coronavirus Pep05. One year post primary immunizations, the mice were given a boost and bled a week after. IgG antibody titers to influenza virus A and IgG responses to human Coronavirus.

Neutralizing titers (day 56) in mice immunized intradermally with lug, 10 μg or 20 μg dose of a vaccine comprising of Coronavirus Pep11 and Coronavirus Pep05. Neutralization of influenza A/Hong Kong (H3N2).

Serum antibody responses in select mice immunized intradermally with 10 μg of a vaccine comprising of Coronavirus Pep11 and Coronavirus Pep05. One year post primary immunizations, the mice were given a boost and bled a week after. IgG1 antibody titers to coronavirus peptides and influenza epitopes.

Serum antibody responses in select mice immunized intradermally with 10 μg of a vaccine comprising of Coronavirus Pep11 and Coronavirus Pep05. One year post primary immunizations, the mice were given a boost and bled a week after. IgG titers to influenza virus A and IgG antibody responses to human Coronavirus.

Neutralizing titers in select mice immunized intradermally with 10 μg of a vaccine comprising of Coronavirus Pep11 and Coronavirus Pep05. Neutralization of influenza A/Hong Kong (H3N2) (ID₇₅ values) is shown one year post primary immunization.

These studies show that serum antibodies induced by both SQ and ID immunization bound to both live influenza and coronavirus and were strongly neutralizing. In addition, mice were immunized with both coronavirus peptide vaccines Pep05 and Pep11 formulated together into a single vaccine. The combined vaccine antigens induced an amazingly robust early response to coronavirus and influenza peptide epitopes and IgG1 bound at very high titers to influenza A strains and to influenza B as well SARSCoV-2 variants. These results are surprising and formulating peptide vaccine antigens together does not cause inhibition of epitope responses, but actually increases immune stimulation and may be related to increased number of immune cells activated. Further studies were done with different routes of administration and using different vaccine antigen doses. Both 1 μg and 10 μg doses induced serum IgG responses that were boosted one year after initial immunization. Robust antibody responses were seen with 1 and 10 μg doses and to include very strong influenza virus neutralization. These data demonstrate that the peptide vaccines can induce a robust immune response when given by various immunization routes.

Vaccine antigens that include HIV and malaria epitopes provide vaccines against important viral and parasitic pathogens. Peptide vaccine antigens provide efficiencies for vaccine production and delivery to populations around the world that often live in rural areas that lack medical infrastructure and require inexpensive vaccines that can provide broad immunity to key pathogens such as HIV and malaria, as well as influenza and tuberculosis.

### Malaria and LPS, Lipooligosaccharide Epitopes

Malaria Epitope (CSP junctional region-
antibody blocks liver invasion)
SEQ ID NO 164:
NPDPNANPNVDPNANGGGC
Lipooligosaccharide mimotopes (nontypeable
H. influenza)
SEQ ID NO 165:
NMMRFTSQPPNNNMMNYIMDPRTH

E. coli and Salmonella LPS Shared Epitopes (Mimotopes)

SEQ ID NO 166:
STLNYMYXAHPF-Core E. coli LPS)
SEQ ID NO 167:
ISLSNIVDSQTP-LPS (S. typhi and E. coli)
SEQ ID NO 168:
GFSVITGAAMFE-LPS core and Lipid A S. urbana
and E. coli)

Combining these and other epitopes provides vaccines and antibodies to important pathogens such as Malaria and HIV, Gram-positive and Gram-negative bacteria in the prevention or treatment of sepsis and shock, H. influenza and Gram-positive vaccine, Malaria, TB and HIV, and Multi-epitope TB vaccine

Combination Microbial Peptides Vaccine Antigens
to Key Pathogens
1. malaria/HIV1/T cell Epitopes-
SEQ ID NO 169:
NPDPNANPNVDPNANGGGCRKSIHLGPGRAFYQYIKANSKFIGITE
2. LPS/LTA/PGN/T Cell epitopes-
SEQ ID NO 170:
STLNYMYXAHPFWRMYFSHRHAHLRSPGGGGGAEKA
QYIKANSKFIGITE
3. LOS H. Flu/PGN/T Cell epitopes-
SEQ ID NO 171:
NMMRFTSQPPNNGGGGGAEKAQYIKANSKFIGITE
4. MALARIA/TB LAM/HIV3/T Cell epitopes-
SEQ ID NO 172:
NPDPNANPNVDPNANGGGCHSFKWLDSPRLRRKSIRIGPGQAFY
QYIKANSKFIGITE
5. PGN/TB LAM and TB 16.3 HSP/T cell epitopes-
SEQ ID NO 173:
AEKAGGGGGHSFKWLDSPRLRSEFAYGSFVRTVSLPVGADEQYIKA
NSKFIGITE

### Example 9 Peptide Vaccines for Targeting the N-Terminal Domain (NTD) of SARS-CoV-2 and Other Coronaviruses

The N-Terminal Domain (NTD) peptide sequences shown in Table 6 could be used to build peptide vaccine antigens with, or without a T-cell epitope (T-cell epitope combined sequences and with one or more coronavirus, influenza, or other microbe epitopes. Highly conserved spike protein receptor binding domain (RBD) epitope with NTD. Coronavirus polymerase epitope with influenza M2e and Neuraminidase (NA) epitopes, combined with NTD:. Coronavirus polymerase with influenza M2e and 2 highly conserved coronavirus spike protein RBD epitopes and a T cell epitope. Coronavirus polymerase with 2 coronavirus highly conserved spike protein RBD epitopes, a coronavirus NTD epitope and a T cell epitope.

TABLE 6
N-Terminal Domain (NTD) of
SARS-CoV-2 and other coronaviruses sequences.

| SEQ | | |
|---|---|---|
| ID | Peptide | |
| NO | number | Peptide Sequence |
| 150 | NTD | CATGIAVAG |
| | Pep01 | |
| 151 | NTD | YYYYYGMDVW |
| | Pep02 | |
| 152 | NTD | CATGYSSSWYFDYW |
| | Pep03 | |
| 153 | NTD | CAKGYSYGYNWFDSW |
| | Pep04 | |
| 154 | NTD | CQQYNNWPPLTF |
| | Pep05 | |
| 155 | NTD | CATGIAVAGQYIKANSKFIGITE |
| | Pep06 | |
| 156 | NTD | YYYYYGMDVWQYIKANSKFIGITE |
| | Pep07 | |
| 157 | NTD | CATGYSSSWYFDYWQYIKANSKFIGITE |
| | Pep08 | |
| 158 | NTD | CAKGYSYGYNWFDSWQYIKANSKFIGITE |
| | Pep09 | |
| 159 | NTD | CQQYNNWPPLTFQYIKANSKFIGITE |
| | Pep10 | |
| 160 | NTD | ARDLICAQCATGYSSSWYFDYWQYIKANS |
| | Pep11 | KFIGITE |
| 161 | NTD | WDYPKCDRATEVETPIRNEHYEECSCYCQ |
| | Pep12 | QYNNWPPLTFQYIKANSKFIGITE |
| 162 | NTD | WDYPKCDRATEVETPIRNEARDLICAQEN |
| | Pep13 | QKLIANCATGIAVAGQYIKANSKDIGITE |
| 163 | NTD | WDYPKCDRAENQKLIANARDLICAQCATG |
| | Pep14 | YSSSWYFDYWQYIKANSKFIGITE |

### Example 10—Peptides and Peptides

To improve vaccine efficiency and global uptake of vaccines peptide vaccine antigens can combine multiple microbial peptide sequences to include, but not limited to peptide sequences that target respiratory viruses, hemorrhagic fever viruses, HIV, parasitic infections like malaria, bacterial infections, such as staphylococcus and M. tuberculosis and fungi such as candida, or aspergillosis. peptide vaccine antigens could include, but are not limited to Malaria and HIV (1), gram negative and gram positive bacteria/toxins (2), gram negative and gram positive bacteria (3), malaria, TB and HIV (4), gram positive bacteria and TB (5). Peptide vaccine antigens can be combined using viral, bacterial, or parasitic peptide sequences in any order with, or without a T cell epitope. In addition, the peptide vaccine antigens can be given individually, or one or more peptide vaccines may be added together to further broaden the microbes targeted. Antibodies that target these peptides would be useful to prevent or treat the microbes that contain the epitopes within the peptides.

### Example 11 IgM Monoclonal Antibodies Targeting Peptidoglycan May Provide Therapeutic Strategies Against Antimicrobial Resistant Bacteria

Antimicrobial resistance (AMR) poses a substantial global threat to human health and development. In addition to death and disability, the cost of AMR to the global economy is significant. Prolonged illness results in longer hospital stays and the need for more expensive medicines and financial challenges for those impacted. Therapeutics such as monoclonal antibodies (mAbs) may offer prevention and control measures against microbial infections without the use of antibiotics. In this study, human antibodies (serum and mAbs) were developed against components of Staphylococcus aureus (SA) and Mycobacterium tuberculosis (MTB) and evaluated their capabilities.

Humanized DRAGA mice were immunized with 20 μg of a combination vaccine comprised of ultrapure peptidoglycan (PGN, derived from SA) and TB Pep01 peptide (targeting MTB HSP16.3), formulated with ADDAVAX™ adjuvant. Serum antibody responses to PGN, TB Pep01, and various whole bacteria were analyzed using ELISA. Mice with high antisera titers was selected for hybridoma production. Hybridomas were screened for binding to PGN, TB Pep01, and whole bacteria using ELISA and high producing clones were selected for monoclonal antibody development. Purified mAb was analyzed for recognition of live bacteria including Mycobacterium smegmatis, Staphylococcus epidermidis, and Staphylococcus aureus. Opsonophagocytic Killing Activity (OPKA) of purified mAb against live mycobacteria was assessed. Humanized DRAGA mice preferentially make IgM antibodies.

IgM monoclonal antibodies targeting peptidoglycan provide therapeutic strategies against antimicrobial resistant bacteria. Profiles of serum antibody responses to PGN and TB Pep 01 was analyzed using IgM and IgG detection antibodies. Day-42 serum antibody responses to MTB CDC1551 is shown. Early and enhanced serum IgM responses to PGN were observed by Day-21, while IgG responses to PGN were detected at Day-35. Antisera binding to TB Pep01 was demonstrated, albeit lower than PGN. In addition, there was antisera recognition of whole bacteria.

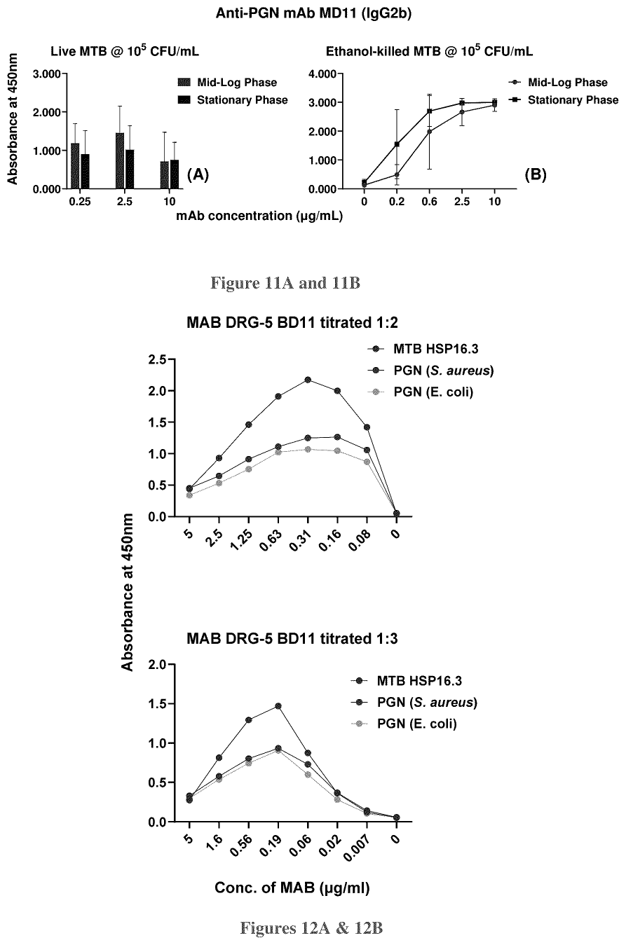

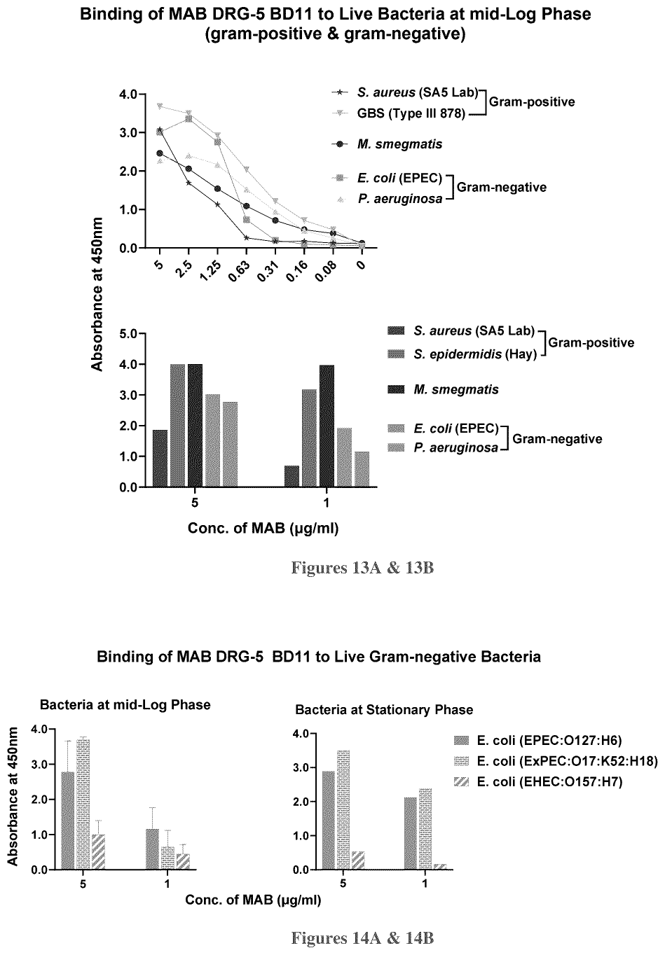

Hybridoma DRG-5 BD11 clones (IgM) targeting PGN were identified for monoclonal antibody production. Purified IgM mAb DRG-5 BD11 bound to ultrapure PGN and to live gram-positive bacteria. Additionally, mAb DRG-5 BD11 bound to MTB HSP16.3 (TB Pep01), and PGN derived from both S. aureus and E. coli demonstrating the bi-specificity of mAB DRG-5 B11 titrated 1:2 (FIG. 12A) and titrated 1:3 (FIG. 12B). MAB DRG-5 BD11 bound to various gram-positive and gram-negative bacteria with live cultures grown to mid-Log phase (FIG. 13A-line graph and FIG. 13B-bar graph). The mAb also recognized and bound to various live E. coli strains (EPEC:0127:H6, ExPEC:017:K52:H18), and EHEC:1157:H7) at both mid-Log (FIG. 14A) and Stationary (FIG. 14B) phases of growth.

Purified IgM mAb DRG-5 BD11 show binding activity to PGN and various live gram-positive bacteria at 10\hat{ }5 CFU/mL and significantly enhanced killing of mycobacteria using U-937 macrophages (Table 7).

TABLE 7
Monoclonal Antibody Functional Activity

| mAb | Peak OPKA |
|---|---|
| 31 | 58% |
| 2 | 50% |
| 1 | 44% |
| 0.5 | 49% |

Preliminary functional activity of human IgM mAb DRG-5 BD11 against mycobacteria (M. smegmatis) showed significant OPKA at 2 μg/mL and 31 μg/mL using U-937 macrophages, which has statistical significance of OPKA >50%.

Hybridomas developed in humanized DRAGA mice immunized with PGN and TBPep01 bound to the immunogens and showed broad recognition of various microbes. Ongoing studies to evaluate bi-specific IgM mAb DRG-5 BD11 functional activity against various microbes to include mycobacteria and staphylococci are in progress. IgM mAbs that recognize and whole bacteria, and opsonize and kill multiple bacterial strains, provide an effective antimicrobial strategy for treatment of drug-resistant bacterial infections.

### Example 12 Antibodies, (Both Polyclonal and Monoclonal) that Provide Both Prophylactic and Therapeutic Treatment Options Against Various Bacteria and the Bacterial Toxins

Many bacteria are becoming increasingly resistant to antibiotics that are essential for treating severe infections such as bacterial pneumonia and sepsis. Peptide vaccines that include multiple highly conserved epitopes, or mimotopes from gram negative (GN) and gram positive (GP) bacteria would be useful for preventing and treating infections caused by these antibiotic-resistant bacteria. In addition, antibodies, (both polyclonal and monoclonal) would provide both prophylactic and therapeutic treatment options against antibiotic resistant bacteria and the bacterial toxins and could be used alone and in combination with antibiotics. Active and passive immunization to prevent wound (trauma related) infections and life-threatening sepsis and shock is of great value in high-risk patients especially those undergoing surgery, or immunosuppressive therapy. Epitopes and mimotopes are selected from a variety of molecules to include PGN, LTA, LPS and LPS core/Lipid A. Different microbial peptide epitopes are combined to produce peptide vaccines (e.g., Table 8). These peptides/vaccines and antibodies (polyclonal and monoclonal) targeting these epitopes are important as bacteria become broadly resistant to many classes of antibiotics.

TABLE 8
LPS Peptides, or mimotopes
that interact with the TLR-4 receptor:

| SEQ | | | | |
|---|---|---|---|---|
| ID NO | | | | |
| | Sequence | Name | Mimotope | Results |
| 174 | QEINSSY | (RS01) | LPS-T | Good cytokine |
| | | | mimotope | induction |
| 175 | APPHALS | (RS09) | LPS | Good cytokine |
| | | | mimotope | induction |
| 176 | VVPTPPY | (RS11) | LPS | Activated |
| | | | mimotope | NF-kB |
| 177 | SMPNPMV | (RS03) | LPS | Activated |
| | | | mimotope | NF-kB |
| 178 | GLQQVLL | (RS04) | LPS | Not very |
| | | | mimotope | soluble |
| 179 | ELAPDSP | (RS12) | LPS | Activated |
| | | | mimotope | NF-kB |

| | Sequence (epitopes) |
|---|---|
| 180 | QEINSSYQYIKANSKFIGITE (LPS-Tetanus |
| | T-cell epitopes) |
| 181 | APPHALSQYIKANSKFIGITE (LPS-Tetanus |
| | T-cell epitope) |
| 182 | VVPTPPYQYIKANSKFIGITE (LPS-Tetanus |
| | T-cell epitope) |
| 183 | QEINSSYAEKAGGGGGWRMYFSHRHAHLRSPQYI |
| | KANSKFIGITE (LPS-PGN-LTA-Tetanus |
| | T-cell epitope) |
| 184 | APPHALSAEKAGGGGGWRMYFSHRHAHLRSPQYI |
| | KANSKFIGITE (LPS-PGN-LTA-Tetanus |
| | T-cell epitope) |
| 185 | VVPTPPYAEKAGGGGGWRMYFSHRHAHLRSPQYI |
| | KANSKFIGITE (LPS-PGN-LTA-Tetanus |
| | T-cell epitope) |
| 186 | AEAKAGGGGGWRMYFSHRHAHLRSPQEINSSYQY |
| | IKANSKFIGITE (PGN-LTA-LPS core/ |
| | Lipid A-Tetanus T-cell epitope) |
| 187 | WRMYFSHRHAHLRSPAPPHALSAEKAGGGGGQYI |
| | KANSKFIGITE (LTA-LPS-PGN-Tetanus |
| | T-cell epitope) |
| 188 | QYIKANSKFIGITEWRMYFSHRHAHLRSAEKAGG |
| | GGGVVPTPPY (Tetanus T-cell- |
| | epitope-LTA-LPS-PGN-LPS) |
| 189 | QEINSSYAEKAGGGGGWRMYFSHRHAHLRSPGFS |
| | VITGAAMFEQYIKANSKFIGITE (LPS-PGN- |
| | LTA-LPS-Tetanus T-cell epitope) |

SEQ ID NO 163 had activated NF-KB with adjuvant. Antibodies that bound to the LPS containing peptides similarly bound to LPS. Peptides RS01 and RS09 were analyzed in BALB/c mice RS09 had adjuvant activity.

### Example 13 Adjuvanted Unconjugated Multi-Epitope Influenza Peptide Vaccine LHNVD-105 Study in Swine

Pigs (n=22) were injected IM with an immunogen comprised of GMP Flu Pep 5906 (SEQ ID NO 81; SLLTEVETPIRNEWGLLTEVETPIRQYIKANSKFIGITE (M1/M2/M2e conserved region with a universal T cell epitope) plus Pep 11 (SEQ ID NO 63); GNLFIAPWGVIHHPHYEECSCY) in ADDAVAX™ reconstituted in water (referred to as LHNVD-105) at 100 μg, 250 μg, or 500 μg, or with PBS as a negative control. Body weight increased from approximately 20 lb to approximately 80 lbs for all animals over the 49-day test period. Body temperatures varied during the test period between approximately 101° F. (39° C.) and approximately 103° F. (40° C.).

Animals were observed throughout the course of the study for any negative local or systemic side effects. Images were taken of injection sites two days post intramuscular immunization. No adverse reactions were observed post immunization in any treatment group. All pigs exhibited normal behavior without signs of distress or reactogenicity at the injection site after administration of the vaccine. Pigs were euthanized at the conclusion of the study.

Binding activity of antisera from pigs immunized with LHNVD-105 at 100 μg, 250 μg, or 500 μg dose or PBS on do and d28 to: (i) whole virus of Flu A/California (H1N1) pdm09; (ii) whole virus of Flu A/Hong Kong/4801/2014 (H3N2); (iii) peptide LHNVD-105. Data are represented as means±SEM. Binding activity increased from 1.0 at OD450 (water) to 1.5 and almost 2.0 for the animals which received LHNVD-105 injections. Surprisingly, antisera from animals that received the lower quantities of peptide (100 μg and 250 μg) showed the greater binding to both whole viruses and the peptide LHNVD-105.

Functional assays were performed of HAI titers on animals injected with LHNVD-105 at 100 μg, 250 μg, or 500 μg dose or PBS on d49 against Flu A/California (H1N1) pdm09 and Flu A/Hong Kong/4801/2014 (H3N2). Once again, antisera from animals administered the lower doses of LHNVD-105 (100 μg and 250 μg) showed a greater HA titer as compared to animals administered the higher dose (500 μg), animals administered PBS only, and pre-immune animals.

### Example 14—Vaccines and Monoclonal Antibodies Targeted to Pathogen Toxins to Prevent Neurodegeneration and Atherosclerosis

Vaccines and Monoclonal Antibodies offer therapeutic and preventive strategies for mitigating the inflammatory processes underlying many inflammatory conditions to include neurodegeneration and atherosclerosis. Vaccine compositions containing one or more epitopes from SARS-CoV-2, or influenza virus or from bacterial toxins such as LPS, PGN, and LTA, designed to stimulate the immune system to produce viral neutralizing antibodies or antibodies that block the interaction between these bacterial toxins and immune receptors like TLR4 and TLR2.

Monoclonal antibodies (Standard or Extended Half-life), alone or in combination engineered to specifically target and neutralize viruses, or LPS, PGN, and LTA, and extended half-life antibodies offer sustained protection by remaining in circulation for extended periods. Antibodies designed with mutations in the Fc region to prolong their half-life by enhancing their interaction with the neonatal Fc receptor (FcRn), which recycles antibodies back into the bloodstream instead of degrading them and other techniques to extend half-life can be used.

The vaccine formulations could include but are not limited to specific epitopes from influenza virus, or bacterial toxins (LPS, PGN, LTA) as well as epitopes such as tetanus toxin, which are known to enhance immunogenicity. These epitopes are designed to mimic the structure of the bacterial components that interact with TLRs, thereby eliciting a strong immune response. The vaccines could also induce the production of neutralizing antibodies that can promote clearance of viruses, or modulate the bacterial toxins interaction with TLRs on immune cells, thereby reducing chronic inflammation in the lung, brain and cardiovascular system.

Antibodies can be designed to target bacterial toxins, such as LPS, PGN, and LTA, with high specificity and affinity. By binding to toxins, the antibodies can modulate the downstream inflammatory responses to prevent or treat sepsis and shock, as well as downstream inflammatory conditions. These antibodies could be administered as a prophylactic measure in individuals at high risk of developing infections or inflammatory conditions to include neurodegenerative diseases or atherosclerosis, or as a therapeutic intervention in those already diagnosed with early-stage disease. In addition, two or more of these monoclonal antibodies could be used together as a cocktail to prevent or treat infections and other antibodies could be added to the formulation to include but not limited to S. aureus alpha toxin, or Beta hemolysin.

Both the vaccines and monoclonal antibodies could act by neutralizing influenza virus, or coronavirus and by neutralizing toxins such as LPS, PGN, and LTA, preventing them from binding to TLR2 and TLR4 on immune cells. This could inhibit the activation of NF-κB and the subsequent release of pro-inflammatory cytokines such as TNF-α, IL-6, and IL-1β, which are key drivers of severe pneumonia, sepsis, neuroinflammation and atherosclerotic plaque formation. In addition, procalcitonin could be used to detect inflammation and sepsis and also used to monitor and guide both when to start and stop therapy. CRP could be used to aid in the detection of influenza and coronavirus infections and guide the therapy to reduce inflammation.

### Example 15—Peptide Formulations with Enhanced Adjuvanting Properties

Peptides can be designed with one or more adjuvanting peptides to provide an internally adjuvanted vaccine and individual peptides/mimotopes can be formulated alone or together with other peptides or individual peptides as a vaccine formulation. Additionally, the peptides may be formulated with peptide and non-peptide adjuvants to enhance the immune response. Adjuvanting peptides such as APPHALS (SEQ ID NO. 175; an LPS peptide/mimotope), can be synthesized in a peptide vaccine with another microbial toxin T cell epitope, such as QYIKANSKFIGITE (SEQ ID NO 61) to be a peptide vaccine that is internally adjuvanted with two adjuvanting peptides that can be given alone, or also formulated with another adjuvant. Cell penetrating peptides (CPP) may also be incorporated into a peptide, or formulated with a peptide vaccine (with or without) an adjuvant to enhance systemic and mucosal immunity when the vaccine is administered orally, or intranasally. In addition, the LPS adjuvanting peptides could be included in a viral peptide vaccine such as SEQ ID NO. 175.

TABLE 9

| SEQ | |
|---|---|
| ID NO | Sequence |
| 190 | GNLFIAPAPPHALSWGVIHHPHYEECSCYQYIKA |
| | NSKFLIGITE |
| 175 | APPHALS (LPS Peptides/Mimotope; |
| | cytokine induction, activates NF-kB) |
| | Flu T cell epitope (specific |
| | influenza T cell epitope) Cell |
| | Penetrating Peptides-Mucosal Immune |
| | Enhancers) |
| 191 | RQIKIWFQNRRMKWKK (Penetratin- |
| | enhances IgA and IgG) |
| 192 | YGRKKRRQRRR (Tat from HIV-1 protein) |
| 193 | HYRIKPTFRRLKWKYKGKFW (limulus) |
| 187 | WRMYFSHRHAHLRSPAPPHALSAEKAGGGGGQYIKA |
| | NSKFIGITE |

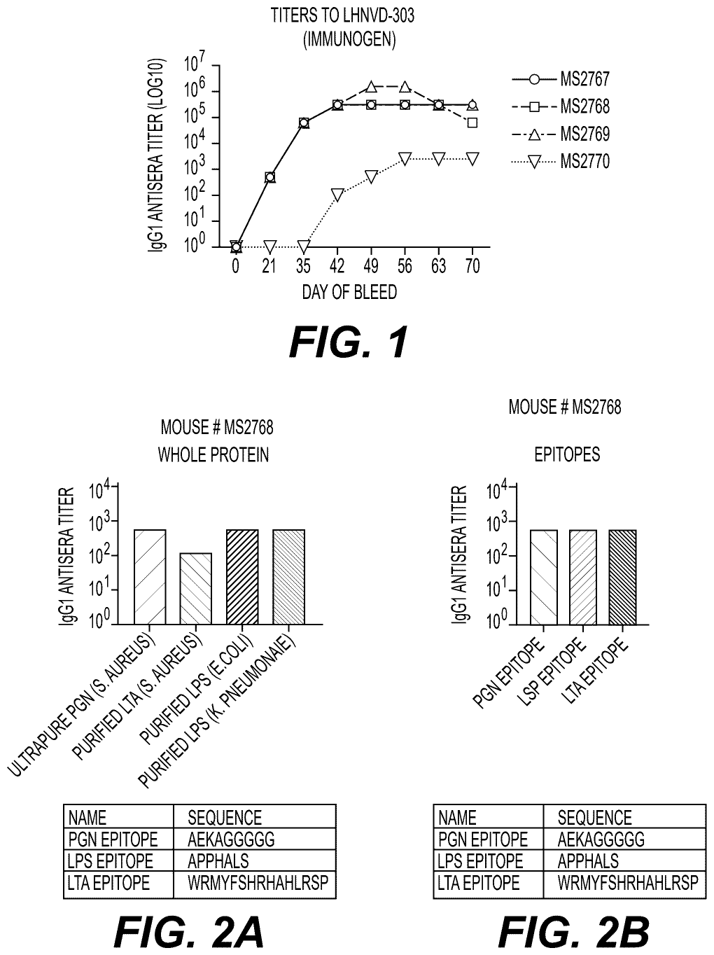

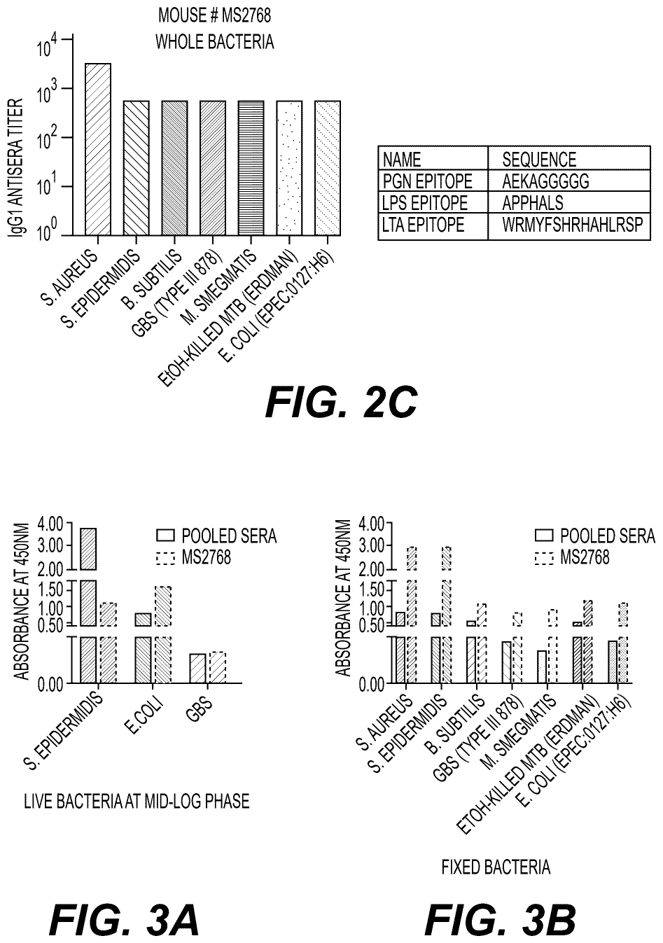

The adjuvanted peptide vaccine that include bacterial toxins induce serum antibodies that bind well to the immunogen (peptide formulation) (see FIG. 1). These peptides that include bacterial toxins induce serum antibodies that bind to gram-positive and gram-negative toxins and to the whole bacteria as well (see FIGS. 2 and 3).

LTA-LPS-PGN-T-Cell Epitope (from tetanus toxin). The LPS mimotope and Tetanus toxin epitopes are each adjuvanting.

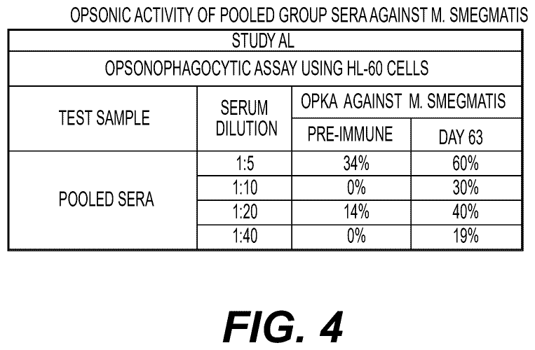

These peptide vaccines targeting bacterial toxins induce antibodies that bind to the toxins and whole bacteria and are designed to both enhance bacterial clearance and neutralize endotoxins to block the effects of the toxins on the brain and other organ systems and also enhance immune eradication of the infection. These peptides that include bacterial toxins generate opsonic antibodies (FIG. 4).

### Example 16—Polymicrobial Peptide Vaccines for Global Immunization, Pandemic Preparedness and Healthy Aging

Peptide vaccine antigen platform includes bacterial and viral epitopes to prevent bacterial and viral infection decrease inflammation and provide immunity across a broad spectrum of microbes, simplify global immunization programs, enhance pandemic preparedness, and decrease chronic inflammation to promote healthy aging. Vaccines could include universal influenza and coronavirus epitopes, and conserved bacterial epitopes and mimotopes such as LPS, PGN, LTA and MTB heat shock protein epitopes to prevent bacterial infection and toxin induced inflammation. The vaccines can also be internally adjuvanted with one or more adjuvanting mimotopes or peptides from LPS, or other toxins such as Tetanus toxin.

TABLE 10
Peptide Sequences

| SEQ ID | |
|---|---|
| NO | Sequence (epitopes) |
| 194 | GNLFIAPWGVIHHPHYEECSCYTEVETPIRNEQY |
| | IKANSKFIGITE |
| | (composite HA epitope; NA epitope; |
| | M2e epitope; T cell epitope) |
| 195 | WDYPKCDRATEVETPIRNEGNLFIAPWGVIHHPH |
| | YEECSCYQYIKANSKFIGITE |
| | (Coronavirus RNA Polymerase; |
| | Matrix (M1/M2/M2e); HA; HA; NA; |
| | Tetanus T-cell epitope) |
| 187 | WRMYFSHRHAHLRSPAPPHALSAEKAGGGGGQYI |
| | KANSKFIGITE |
| | (LTA; LPS; PGN; Tetanus T-cell |
| | epitope) |
| 196 | SEFAYGSFVRTVSLPVGADEWRMYFSHRHAHLRS |
| | PAPPHALSAEKAGGQYIKANSKFIGITE |
| | (TB 16 kD Heat Shock Protein; LTA; |
| | LPS; PGN; Tetanus T-cell epitope) |
| 197 | SEFAYGSFVRTVSLPVGADETEVETPIRNEGNLF |
| | IAPWGVIHHPHYEECSCYQYIKANSKFIGITE |
| | (TB 16 kD Heat Shock Protein; |
| | Matrix (M1/M2/M2e); HA; HA; NA; |
| | Tetanus T-cell epitope) |
| 198 | GNLFIAPWGVIHHPHYEECSCYTEVETPIRNEQY |
| | IKANSKFIGITEAPPHALS |
| | (HA; HA; NA; Matrix (M1/M2/M2e); |
| | Tetanus T-cell epitope; LPS) |
| 199 | APPHALSGNLFIAPWGVIHHPHYEECSCYTEVET |
| | PIRNEQYIKANSKFIGITE |
| | (LPS; HA; HA; NA; Matrix (M1/ |
| | M2/M2e); Tetanus T-cell epitope) |
| 200 | GNLFIAPWGVIHHPHYEECSCYAPPHALSTEVET |
| | PIRNEQYIKANSKFIGITE |
| | (HA; HA; NA; LPS; Matrix (M1/M2/ |
| | M2e); Tetanus T-cell epitope) |
| | Using an influenza T-cell epitope |
| | from NP provides a different, or |
| | an additional T-cell stimulation |
| SEQ ID | TYQRTRALV (NP T-cell epitope) |
| NO 201 | |
| SEQ ID | TYQRTRALVGNLFIAPWGVIHHPHYEECSCYTEV |
| NO 202 | ETPIRNEQYIKANSKFIGITE |
| SEQ ID | GNLFIAPWGVIHHPHYEECSCYTEVETPIRNETY |
| NO 203 | QRTRALV |
| SEQ ID | WDYPKCDRATEVETPIRNEGNLFIAPWGVIHHPH |
| NO 204 | YEECSCYQYIKANSKFIGITE |
| | (Cor RNA pol epitope plus Inf M1/ |
| | M2e epitope plus Inf HA composite |
| | epitope plus Inf NA epitope plus |
| | Tetanus T cell epitope) |

### Example 17—Procalcitonin as a Biomarker that Determines a Severity of Systemic Inflammation or Bacterial Infection in the Subject

Procalcitonin (PCT) is a 116 amino acid precursor of calcitonin is normally produced by the thyroid C-cells. Serum concentrations of PCT are normally less than 0.05 ng/ml. In circumstances of systemic inflammation, particularly bacterial infection, PCT is produced in large quantities by different tissues. Elevated PCT levels are detectable within 2 to 4 hours an infection and peak within 6-24 hours, and not otherwise effected by an immunosuppressive state. PCT levels parallel the severity of the inflammatory insult or infection meaning those with more severe disease have higher levels, and can be a prognostic indicator with higher serum concentrations related to the risk of morbidity and mortality.

PCT and calcitonin levels are markedly upregulated in response to microbial toxins such as lipopolysaccharide (LPS) from Gram-negative bacteria and lipoteichoic acid (LTA) from Gram-positive bacteria. While initially characterized as a biomarker for bacterial sepsis, PCT is now recognized as a sensitive and dynamic indicator of systemic exposure to bacterial components, even in the absence of overt infection.

PCT level has been used to assist clinicians in antibiotic management of patients with lower respiratory tract infections such as pneumonia and bronchitis. An analysis of eight clinical studies involving over three thousand patients found the use of PCT resulted in an almost one third decrease in antibiotic prescriptions with a parallel decrease in antibiotic duration. Clinical trials performed evaluating the utility of PCT levels in guiding antibiotic therapy show a decrease in antimicrobial exposure of 19-38% without increases in mortality, length of stay, or relapsed/persistent infection. Most studies in sepsis have evaluated using PCT to discontinue antibiotics although one large trial did use PCT levels to assist in the decision to initiate treatment.7 Because of limited data, the decision to initiate therapy in the ICU should be driven by the severity of illness and clinical assessment of the likelihood of infection with the PCT used as an adjunct to assist in the decision to initiate antibiotics. Much more rigorous evidence exists to support the use of PCT to discontinue antibiotics. Recommendations for patients considered at risk for bacterial infection based on PCT levels are shown in Table 11.

TABLE 11

| | |
|---|---|
| PCT Level (μg/L) | Recommendation |

Patients with Lower Respiratory Tract Infections

| | |
|---|---|
| Less than 0.1 | Antibiotics discouraged |
| 0.1 to 0.24 | Antibiotics not recommended |
| 0.25 to 0.5 | Antibiotics suggested |
| Greater than 0.5 | Antibiotics encouraged |

Patients with Sepsis

| | |
|---|---|
| Less than 0.25 | Antibiotics discouraged |
| 0.25 to 0.5 | Antibiotics not recommended |
| 0.5 to 1.0 | Antibiotics suggested |
| 1.0 to 10.0 | Antibiotics encouraged |
| Greater than 10.0 | Emergency treatment due to septic shock |

Thus, PCT levels are strong prognostic indicators of lack of control of the infection by the patient's own immune system and/or prescribed antibiotic treatment. In both patients with lower respiratory infections or patients with sepsis, rising PCT values can be used to indicate that an existing treatment is not having the desired effect and that a more aggressive treatment is needed.

The elevation of PCT reflects the presence of microbial-associated molecular patterns (MAMPs) that activate innate immune receptors and trigger a proinflammatory cascade. Further, elevated PCT levels can be detected in non-infectious chronic conditions, indicated the presence of a low-grade microbial infection with subdued immune activation.

This microbial burden plays a causal and amplifying role in a wide range of inflammatory diseases. Chronic exposure to LPS, LTA, and peptidoglycan (PGN) has been mechanistically linked to the progression of atherosclerosis, type 2 diabetes, non-alcoholic fatty liver disease (NAFLD), chronic kidney disease, and neurodegenerative diseases such as Alzheimer's disease, where microbial products have been found in cerebrovascular tissues and plaques. In the gut, these toxins contribute to inflammatory bowel disease (IBD) by disrupting epithelial integrity and perpetuating mucosal immune activation. In the joints, they have been detected in the synovial fluid of patients with rheumatoid arthritis, suggesting a systemic route of dissemination. Furthermore, chronic low-level microbial activation of toll-like receptors has been implicated in depression, cognitive decline, and sarcopenia, underscoring the systemic reach of these inflammatory triggers. Thus, PCT levels can be used to differentiate between conditions such as such as sepsis vs. respiratory infection vs. chronic low-level infections.

By reducing PCT levels, one can lower a quantifiable biochemical marker of microbial burden, and also interrupt the inflammatory signaling loops that sustain or exacerbate these chronic diseases. Generating or administering neutralizing antibodies to LPS, LTA, and PGN, targets the upstream drivers of this biochemical and immunological disturbance. Doing so serves as a core therapeutic platform for reducing systemic inflammation, preserving physiological function, and preventing or attenuating disease progression across a broad spectrum of age-related conditions.

As with all diagnostic measures, false positive and false negative can occur and clinical examination should be included. For example, PCT elevations may be due to a non-bacterial cause such as in newborns, patients under stress, patients currently being treated with agents that stimulate cytokines (e.g., OKT3, anti-lymphocyte globulins, alemtuzumab, IL-2, granulocyte transfusion), patients currently infected with P. falciparum, patients under cardiogenic shock or organ perfusion abnormalities, patients having graft vs. host disease, patients with small cell lung cancer, patients with compromised renal function. All these conditions are identifiable, such that PCT levels can be determined and a diagnosis made in patients not under such risks.

### Example 18—PANDAS

Pediatric Autoimmune Neuropsychiatric Disorders Associated with Streptococcal infections (PANDAS), is a condition that can affect children after an infection such as strep throat or scarlet fever, and is a form of pediatric acute-onset neuropsychiatric syndrome (PANS). PANDAS is characterized by an often-sudden onset of neuropsychiatric symptoms in the child, such as, for example, obsessive-compulsive disorder (OCD), mood swings, and anxiety, along with potential deterioration in school performance and eating habits. Presently there is no conclusive blood test for PANDAS/PANS.

PANDAS is believed to be triggered by this inflammatory response and the body's immune system attacking the brain in response to a streptococcal infection. During a bacterial infection, bacterial organisms and bacterial toxins such as LTA and PGN induced inflammation locally and systemically. The immune system produces antibodies to fight an infection, while those same antibodies attack healthy cells in other tissues, in particular brain tissue that mimic epitopes of the strep infection. This is referred to as autoimmunity and, at the cellular level, autoimmunity is characterized by the presence of antibodies or T cells that attack otherwise healthy tissue. It is believed that people generally have low levels of microbial inflammation, but inflammation and autoimmune diseases may be diagnosed when inflammation induces an attack on healthy tissue resulting in physiological changes in the body, which many manifest as neurological. These attacks are believed to lead to the psychological and neurological symptoms associated with PANDAS.

Other embodiments and uses of the invention will be apparent to those skilled in the art from consideration of the specification and practice of the invention disclosed herein. All references cited herein, including all publications and U.S. and foreign patents and patent applications, are specifically and entirely incorporated by reference including U.S. Patent Publication No. 20210246174A1 entitled “Immunogenic Compositions to Treat and Prevent Microbial Infections”, published Aug. 12, 2021, U.S. Pat. No. 9,821,047 entitled “Enhancing Immunity to Tuberculosis,” which issued Nov. 21, 2017, U.S. Pat. No. 9,598,462 entitled “Composite Antigenic Sequences and Vaccines” which issued Mar. 21, 2017, U.S. Pat. No. 10,004,799 entitled “Composite Antigenic Sequences and Vaccines” which issued Jun. 26, 2018, U.S. Pat. No. 8,652,782 entitled “Compositions and Method for Detecting, Identifying and Quantitating Mycobacterial-Specific Nucleic Acid,” which issued Feb. 18, 2014, U.S. Pat. No. 9,481,912 entitled “Compositions and Method for Detecting, Identifying and Quantitating Mycobacterial-Specific Nucleic Acid,” which issued Nov. 1, 2016, U.S. Pat. No. 8,821,885 entitled “Immunogenic Compositions and Methods,” which issued Sep. 2, 2014, and all corresponding U.S. Provisional and continuation applications relating to any of the foregoing patents. The term comprising, wherever used, is intended to include the terms consisting of, and consisting essentially of. Furthermore, the terms comprising, including, containing and the like are not intended to be limiting. It is intended that the specification and examples be considered exemplary only with the true scope and spirit of the invention indicated by the following claims.

1. A composition comprising a peptide sequence containing a combination of epitopes and/or mimotopes from a plurality of the molecules selected from the group consisting of peptidoglycan (PGN), lipoteichoic acid (LTA), and lipopolysaccharide (LPS).

2. The composition of claim 1, wherein the PGN is obtained or derived from a gram-positive, or gram-negative microorganism, or Mycobacteria.

3. The composition of claim 2, wherein the gram-positive microorganism comprises one or more species belonging to genera including Mycobacteria, Staphylococcus, Bacillus, and Streptococcus.

4. The composition of claim 2, wherein the gram-negative microorganism comprises one or more species belonging to genera including Escherichia and Pseudomonas.

5. The composition of claim 2, wherein the mycobacteria comprise one or more species including Mycobacterium tuberculosis and Mycobacterium smegmatis.

6. The composition of claim 1, wherein the LTA is obtained or derived from a gram-positive microorganism.

7. The composition of claim 6, wherein the gram-positive microorganism is of a spp. of Staphylococcus, a spp. of Bacillus, or a spp. of Streptococcus.

8. The composition of claim 1, wherein the LPS is synthetically derived.

9. The composition of claim 8, wherein the gram-negative microorganism is an enteric or respiratory pathogen.

10. The composition of claim 1, comprising epitopes and/or mimotopes from each of the molecules PGN, LTA, and LPS.

11. The composition of claim 1, further comprising an epitope of a heat shock protein (HSP).

12. The composition of claim 1, further comprising an epitope of a lipoarabinomannan (LAM).

13. The composition of claim 1, further comprising two or more T cell stimulating epitope or mimotope.

14. The composition of claim 13, wherein the T cell stimulating epitopes are obtained or derived from tetanus toxin, tetanus toxin heavy chain proteins, diphtheria toxoid, CRM, recombinant CRM, tetanus toxoid, Pseudomonas exoprotein A, Pseudomonas aeruginosa toxoid, Bordetella pertussis toxoid, Clostridium perfringens toxoid, Gram negative bacteria LPS, Escherichia coli heat-labile toxin B subunit, Neisseria meningitidis outer membrane complex, Hemophilus influenzae protein D, Flagellin Fli C, Horseshoe crab Haemocyanin, and/or a fragment, derivative, or modification thereof.

15. The composition of claim 1, further comprising an added adjuvant.

16. The composition of claim 15, wherein the adjuvant comprises a toll-like receptor agonist (TLR), Freund's adjuvant, ALFQ, ALFQA, ALFA, AS01, AS01b, a liposome adjuvant, saponin, lipid A, squalene, and/or modifications, emulsions, nano-emulsions, derivatives, and combinations thereof.

17. The composition of claim 1, which treats or prevents neurodegeneration of mammalian brain tissue.

18. A vaccine comprising the composition of claim 1.

19. A composition comprising one or more of antibodies that bind to the composition of claim 1.

20. The composition of claim 19, containing two or more antibodies.

21. The antibodies of claim 19, which comprises IgG, IgA, IgD, IgE, IgM, or fragments or combinations thereof.

22. The antibodies of claim 19, which induce opsonophagocytic activity and specifically bind to toxins.

23. The antibodies of claim 19, which are polyclonal, bifunctional, bispecific, and/or monoclonal.

24. The antibodies of claim 19, which are fully human or humanized.

25. The antibodies of claim 19, which are modified to extend half-life of the antibodies.

26. A hybridoma that expresses an antibody of claim 19.

27. A method for prevention and/or treatment of a neurodegeneration of mammalian brain tissue comprising administering a peptide sequence containing a combination of epitopes and/or mimotopes of peptidoglycan (PGN), lipoteichoic acid (LTA), and/or lipopolysaccharide (LPS), or antibodies that are reactive to epitopes or mimotopes of peptidoglycan (PGN), lipoteichoic acid (LTA), and/or lipopolysaccharide (LPS).

28. The method of claim 27, wherein the neurodegeneration of mammalian brain tissue is Alzheimer's disease, frontotemporal dementia, chronic traumatic encephalopathy (CTE), Lewy body dementia and/or limbic predominant age-related TDP-43 encephalopathy (LATE).

29. The method of claim 27, which prevents the accumulation of amyloid-beta particles (Aβ) in mammalian brain tissue.

30. The method of claim 27, wherein administration is oral, sub-cutaneous, intra-muscular, intradermal, or intra-nasal.

31. The method of claim 30, wherein administration produces antibodies that provide an opsonophagocytic response and enhance clearance of bacterial toxins present in the subject.

32. A method for prevention and/or treatment of an abnormal proliferation of cells comprising administering a peptide sequence containing a combination of epitopes and/or mimotopes of peptidoglycan (PGN), lipoteichoic acid (LTA), and/or lipopolysaccharide (LPS), or antibodies that are reactive to epitopes or mimotopes of peptidoglycan (PGN), lipoteichoic acid (LTA), and/or lipopolysaccharide (LPS) to a subject.

33. The method of claim 32, wherein the abnormal proliferation is a cancer, a tumor, or an inflammatory disorder.

34. The method of claim 33, wherein the cancer is breast, prostate, bladder, lung, or pancreatic cancer.

35. The method of claim 32, wherein administration is oral, sub-cutaneous, intra-muscular, intradermal, or intra-nasal.

36. The method of claim 32, wherein administration produces antibodies that provide an opsonophagocytic response and enhance clearance of bacterial toxins present in the subject.

37. A method for prevention and/or treatment of an inflammation comprising administering a peptide sequence containing a combination of epitopes and/or mimotopes of peptidoglycan (PGN), lipoteichoic acid (LTA), and/or lipopolysaccharide (LPS), or antibodies that are reactive to epitopes or mimotopes of peptidoglycan (PGN), lipoteichoic acid (LTA), and/or lipopolysaccharide (LPS) to a subject.

38. The method of claim 37, wherein the inflammation comprises chronic inflammation and disorders related to chronic inflammation.

39. A method for prevention and/or treatment of sepsis comprising a peptide sequence containing a combination of epitopes and/or mimotopes of peptidoglycan (PGN), lipoteichoic acid (LTA), and/or lipopolysaccharide (LPS), or antibodies that are reactive to a plurality of epitopes or mimotopes of peptidoglycan (PGN), lipoteichoic acid (LTA), and/or lipopolysaccharide (LPS).

40. A method for prevention and/or treatment of a neurodegeneration of mammalian brain tissue in a subject comprising: administering a peptide sequence containing a combination of epitopes and/or mimotopes from a plurality of the molecules selected from the group consisting of peptidoglycan (PGN), lipoteichoic acid (LTA), and lipopolysaccharide (LPS), or antibodies that are reactive to a plurality of epitopes or mimotopes of peptidoglycan (PGN), lipoteichoic acid (LTA), and/or lipopolysaccharide (LPS); and monitoring procalcitonin in the blood of the subject, wherein a serum concentration of at least 0.5 ng/ml is indicative of neurodegeneration.

41. The method of claim 40, wherein administration of the peptide or the antibodies is continued or adjusted based on the level of procalcitonin in the subject.

42. The method of claim 41, wherein the procalcitonin is a biomarker that determines a severity of systemic inflammation or bacterial infection in the subject.

43. The method of claim 41, further comprising monitoring procalcitonin levels post-administration to determine a therapeutic efficacy of the peptide or collection of antibodies.

44. The method of claim 40, further comprising monitoring C-reactive protein (CRP) levels in the blood of the subject.

45. The method of claim 44, wherein procalcitonin is used to differentiate between bacterial and viral etiologies to determine appropriateness of therapy with anti-bacterial toxin immunological compositions.


SEQUENCE LISTING (204 sequences)

SEQ ID NO: 1 - protein, 19 aa, synthetic construct
DWSGYSGSFVQHPELTGLD

SEQ ID NO: 2 - protein, 7 aa, synthetic construct
ETPIRNE

SEQ ID NO: 3 - protein, 15 aa, synthetic construct
FVIREPFISCSHLEC

SEQ ID NO: 4 - protein, 6 aa, synthetic construct
GNFIAP

SEQ ID NO: 5 - protein, 6 aa, synthetic construct
GNLIAP

SEQ ID NO: 6 - protein, 7 aa, synthetic construct
GNLFIAP

SEQ ID NO: 7 - protein, 7 aa, synthetic construct
GNLIFAP

SEQ ID NO: 8 - protein, 8 aa, synthetic construct
HYEECSCY

SEQ ID NO: 9 - protein, 10 aa, synthetic construct
LLTEVETPIR

SEQ ID NO: 10 - protein, 11 aa, synthetic construct
LLTEVETPIRN

SEQ ID NO: 11 - protein, 12 aa, synthetic construct
LLTEVETPIRNE

SEQ ID NO: 12 - protein, 18 aa, synthetic construct
DWSGYSGSFVQHPELTGL

SEQ ID NO: 13 - protein, 9 aa, synthetic construct
EVETPIRNE

SEQ ID NO: 14 - protein, 38 aa, synthetic construct
FLLPEDETPIRNEWGLLTDDETPIRYIKANSKFIGITE

SEQ ID NO: 15 - protein, 64 aa, synthetic construct
GNLFIAPGNLFIAPHYEECSCYHYEECSCYQYIKANSKFIGITEHYEECSCYTPIRNETPIRNE

SEQ ID NO: 16 - protein, 35 aa, synthetic construct
GNLFIAPGNLFIAPQYIKANSKFIGITEGNLFIAP

SEQ ID NO: 17 - protein, 48 aa, synthetic construct
HYEECSCYDWSGYSGSFVQHPELTGLHYEECSCYQYIKANSKFIGITE

SEQ ID NO: 18 - protein, 33 aa, synthetic construct
ITGFAPFSKDNSIRLSAGGDIWVTREPYVSCDP

SEQ ID NO: 19 - protein, 7 aa, synthetic construct
IWGIHHP

SEQ ID NO: 20 - protein, 7 aa, synthetic construct
IWGVHHP

SEQ ID NO: 21 - protein, 8 aa, synthetic construct
IWGVIHHP

SEQ ID NO: 22 - protein, 8 aa, synthetic construct
IWGIVHHP

SEQ ID NO: 23 - protein, 16 aa, synthetic construct
KSCINRCFYVELIRGR

SEQ ID NO: 24 - protein, 27 aa, synthetic construct
LLTEVETPIRNESLLTEVETPIRNEWG

SEQ ID NO: 25 - protein, 13 aa, synthetic construct
LLTEVETPIRNEW

SEQ ID NO: 26 - protein, 14 aa, synthetic construct
LLTEVETPIRNEWG

SEQ ID NO: 27 - protein, 11 aa, synthetic construct
LTEVETPIRNE

SEQ ID NO: 28 - protein, 12 aa, synthetic construct
LTEVETPIRNEW

SEQ ID NO: 29 - protein, 13 aa, synthetic construct
LTEVETPIRNEWG

SEQ ID NO: 30 - protein, 9 aa, synthetic construct
MSLLTEVET

SEQ ID NO: 31 - protein, 10 aa, synthetic construct
MSLLTEVETP

SEQ ID NO: 32 - protein, 11 aa, synthetic construct
MSLLTEVETPI

SEQ ID NO: 33 - protein, 12 aa, synthetic construct
MSLLTEVETPIR

SEQ ID NO: 34 - protein, 13 aa, synthetic construct
MSLLTEVETPIRN

SEQ ID NO: 35 - protein, 14 aa, synthetic construct
MSLLTEVETPIRNE

SEQ ID NO: 36 - protein, 20 aa, synthetic construct
MSLLTEVETPIRNETPIRNE

SEQ ID NO: 37 - protein, 15 aa, synthetic construct
MSLLTEVETPIRNEW

SEQ ID NO: 38 - protein, 16 aa, synthetic construct
MSLLTEVETPIRNEWG

SEQ ID NO: 39 - protein, 24 aa, synthetic construct
MSLLTEVETPIRNEWGCRCNDSSD

SEQ ID NO: 40 - protein, 8 aa, synthetic construct
SLLTEVET

SEQ ID NO: 41 - protein, 11 aa, synthetic construct
SLLTEVETPIR

SEQ ID NO: 42 - protein, 13 aa, synthetic construct
SLLTEVETPIRNE

SEQ ID NO: 43 - protein, 14 aa, synthetic construct
SLLTEVETPIRNEW

SEQ ID NO: 44 - protein, 15 aa, synthetic construct
SLLTEVETPIRNEWG

SEQ ID NO: 45 - protein, 21 aa, synthetic construct
SLLTEVETPIRNEWGTPIRNE

SEQ ID NO: 46 - protein, 27 aa, synthetic construct
SLLTEVETPIRNEWGTPIRNETPIRNE

SEQ ID NO: 47 - protein, 33 aa, synthetic construct
SLLTEVETPIRNEWGTPIRNETPIRNETPIRNE

SEQ ID NO: 48 - protein, 25 aa, synthetic construct
SLLTEVETPIRNEWGLLTEVETPIR

SEQ ID NO: 49 - protein, 10 aa, synthetic construct
TEVETPIRNE

SEQ ID NO: 50 - protein, 6 aa, synthetic construct
TPIRNE

SEQ ID NO: 51 - protein, 8 aa, synthetic construct
VETPIRNE

SEQ ID NO: 52 - protein, 52 aa, synthetic construct
VTREPYVSCDPKSCINRCFYVELIRGRVTREPYVSCDPWYIKANSKFIGITE

SEQ ID NO: 53 - protein, 6 aa, synthetic construct
WGIHHP

SEQ ID NO: 54 - protein, 6 aa, synthetic construct
WGVHHP

SEQ ID NO: 55 - protein, 7 aa, synthetic construct
WGVIHHP

SEQ ID NO: 56 - protein, 7 aa, synthetic construct
WGIVHHP

SEQ ID NO: 57 - protein, 8 aa, synthetic construct
YIWGIHHP

SEQ ID NO: 58 - protein, 8 aa, synthetic construct
YIWGVHHP

SEQ ID NO: 59 - protein, 9 aa, synthetic construct
YIWGVIHHP

SEQ ID NO: 60 - protein, 9 aa, synthetic construct
YIWGIVHHP

SEQ ID NO: 61 - protein, 14 aa, synthetic construct
QYIKANSKFIGITE

SEQ ID NO: 62 - protein, 15 aa, synthetic construct
PIRNEWGCRCNDSSD

SEQ ID NO: 63 - protein, 22 aa, synthetic construct
GNLFIAPWGVIHHPHYEECSCY

SEQ ID NO: 64 - protein, 10 aa, synthetic construct
CAGAGNFIAP

SEQ ID NO: 65 - protein, 10 aa, synthetic construct
CAGAGNLIAP

SEQ ID NO: 66 - protein, 11 aa, synthetic construct
CAGAGNLFIAP

SEQ ID NO: 67 - protein, 10 aa, synthetic construct
CAGAWGVHHP

SEQ ID NO: 68 - protein, 10 aa, synthetic construct
CAGAWGIHHP

SEQ ID NO: 69 - protein, 11 aa, synthetic construct
CAGAWGVIHHP

SEQ ID NO: 70 - protein, 11 aa, synthetic construct
CAGAWGIVHHP

SEQ ID NO: 71 - protein, 13 aa, synthetic construct
GNLIAPWGVIHHP

SEQ ID NO: 72 - protein, 17 aa, synthetic construct
CAGAGNLIAPWGVIHHP

SEQ ID NO: 73 - protein, 14 aa, synthetic construct
GNLFIAPWGVIHHP

SEQ ID NO: 74 - protein, 18 aa, synthetic construct
CAGAGNLFIAPWGVIHHP

SEQ ID NO: 75 - protein, 12 aa, synthetic construct
CAGAHYEECSCY

SEQ ID NO: 76 - protein, 26 aa, synthetic construct
CAGAGNLFIAPWGVIHHPHYEECSCY

SEQ ID NO: 77 - protein, 28 aa, synthetic construct
GNLFIAPWGVIHHPGNLFIAPWGVIHHP

SEQ ID NO: 78 - protein, 32 aa, synthetic construct
CAGAGNLFIAPWGVIHHPGNLFIAPWGVIHHP

SEQ ID NO: 79 - protein, 22 aa, synthetic construct
HYEECSCYGNLFIAPWGVIHHP

SEQ ID NO: 80 - protein, 22 aa, synthetic construct
GNLFIAPHYEECSCYWGVIHHP

SEQ ID NO: 81 - protein, 39 aa, synthetic construct
SLLTEVETPIRNEWGLLTEVETPIRQYIKANSKFIGITE

SEQ ID NO: 82 - protein, 52 aa, synthetic construct
VTREPYVSCDPKSCINRCFYVELIRGRVTREPYVSCDPQYIKANSKFIGITE

SEQ ID NO: 83 - protein, 12 aa, synthetic construct
GNLFIAPRYAFA

SEQ ID NO: 84 - protein, 16 aa, synthetic construct
CAGAGNLFIAPRYAFA

SEQ ID NO: 85 - protein, 11 aa, synthetic construct
GNLVVPRYAFA

SEQ ID NO: 86 - protein, 15 aa, synthetic construct
CAGAGNLVVPRYAFA

SEQ ID NO: 87 - protein, 11 aa, synthetic construct
GNLIAPRYAFA

SEQ ID NO: 88 - protein, 15 aa, synthetic construct
CAGAGNLIAPRYAFA

SEQ ID NO: 89 - protein, 6 aa, synthetic construct
GNLVVP

SEQ ID NO: 90 - protein, 10 aa, synthetic construct
CAGAGNLVVP

SEQ ID NO: 91 - protein, 19 aa, synthetic construct
CAGAFVIREPFISCSHLEC

SEQ ID NO: 92 - protein, 36 aa, synthetic construct
GNLFIAPWGVIHHPHYEECSCYQYIKANSKFIGITE

SEQ ID NO: 93 - protein, 36 aa, synthetic construct
QYIKANSKFIGITEGNLFIAPWGVIHHPHYEECSCY

SEQ ID NO: 94 - protein, 46 aa, synthetic construct
QYIKANSKFIGITEGNLFIAPWGVIHHPHYEECSCYTEVETPIRNE

SEQ ID NO: 95 - protein, 7 aa, synthetic construct
HVEECSY

SEQ ID NO: 96 - protein, 6 aa, synthetic construct
WFIHHP

SEQ ID NO: 97 - protein, 11 aa, synthetic construct
DLWSYNAELLV

SEQ ID NO: 98 - protein, 11 aa, synthetic construct
DIWTYNAELLV

SEQ ID NO: 99 - protein, 7 aa, synthetic construct
YPKCDRA

SEQ ID NO: 100 - protein, 9 aa, synthetic construct
WDYPKCDRA

SEQ ID NO: 101 - protein, 23 aa, synthetic construct
SLDQINVTFLDLEYEMKKLEESY

SEQ ID NO: 102 - protein, 37 aa, synthetic construct
SLDQINVTFLDLEYEMKKLEESYQYIKANSKFIGITE

SEQ ID NO: 103 - protein, 23 aa, synthetic construct
WDYPKCDRAQYIKANSKFIGITE

SEQ ID NO: 104 - protein, 46 aa, synthetic construct
WDYPKCDRASLDQINVTFLDLEYEMKKLEESYQYIKANSKFIGITE

SEQ ID NO: 105 - protein, 41 aa, synthetic construct
WDYPKCDRATEVETPIRNEHYEECSCYQYIKANSKFIGITE

SEQ ID NO: 106 - protein, 8 aa, synthetic construct
ARDLICAQ

SEQ ID NO: 107 - protein, 14 aa, synthetic construct
KWPWYIWLGFIAGL

SEQ ID NO: 108 - protein, 8 aa, synthetic construct
ENQKLIAN

SEQ ID NO: 109 - protein, 30 aa, synthetic construct
ARDLICAQKWPWYIWLGFIAGLENQKLIAN

SEQ ID NO: 110 - protein, 16 aa, synthetic construct
ENQKLIANARDLICAQ

SEQ ID NO: 111 - protein, 25 aa, synthetic construct
WDYPKCDRAENQKLIANARDLICAQ

SEQ ID NO: 112 - protein, 31 aa, synthetic construct
WDYPKCDRAENQKLIANKWPWYIWLGFIAGL

SEQ ID NO: 113 - protein, 39 aa, synthetic construct
ARDLICAQENQKLIANWDYPKCDRAQYIKANSKFIGITE

SEQ ID NO: 114 - protein, 76 aa, synthetic construct
KWPWYIWLGFIAGLWDYPKCDRAQYIKANSKFIGITEARDLICAQENQKLIANWDYPKCDRAQYIKANSKFIGITE

SEQ ID NO: 115 - protein, 69 aa, synthetic construct
ARDLICAQENQKLIANQYIKANSKFIGITEARDLICAQENQKLIANWDYPKCDRAQYIKANSKFIGITE

SEQ ID NO: 116 - protein, 80 aa, synthetic construct
WDYPKCDRATEVETPIRNEHYEECSCYQYIKANSKFIGITEARDLICAQENQKLIANWDYPKCDRAQYIKANSKFIGITE

SEQ ID NO: 117 - protein, 39 aa, synthetic construct
HYEECSCYWDYPKCDRAVETPIRNEQYIKANSKFIGITE

SEQ ID NO: 118 - protein, 40 aa, synthetic construct
ENQKLIANTEVETPIRNEHYEECSCYQYIKANSKFIGITE

SEQ ID NO: 119 - protein, 13 aa, synthetic construct
AEKAGGGGGAEKA

SEQ ID NO: 120 - protein, 19 aa, synthetic construct
AEKAEKAGGGGGAEKAEKA

SEQ ID NO: 121 - protein, 26 aa, synthetic construct
QYIKANSKFIGITEAEKAGGGGAEKA

SEQ ID NO: 122 - protein, 27 aa, synthetic construct
AEKAGGGGGAEKAQYIKANSKFIGITE

SEQ ID NO: 123 - protein, 4 aa, synthetic construct
AEKA

SEQ ID NO: 124 - protein, 9 aa, synthetic construct
AEKAGGGGG

SEQ ID NO: 125 - protein, 5 aa, synthetic construct
GGGGG

SEQ ID NO: 126 - protein, 20 aa, synthetic construct
SEFAYGSFVRTVSLPVGADE

SEQ ID NO: 127 - protein, 42 aa, synthetic construct
SEFAYGSFVRTVSLPVGADEGNLFIAPWGVIHHPHYEECSCY

SEQ ID NO: 128 - protein, 12 aa, synthetic construct
HSFKWLDSPRLR

SEQ ID NO: 129 - protein, 12 aa, synthetic construct
ISLTEWSMWYRH

SEQ ID NO: 130 - protein, 15 aa, synthetic construct
WRMYFSHRHAHLRSP

SEQ ID NO: 131 - protein, 15 aa, synthetic construct
WHWRHRIPLQLAAGR

SEQ ID NO: 132 - protein, 22 aa, synthetic construct
GNLFIAPWGVIHHPHYEECSCY

SEQ ID NO: 133 - protein, 20 aa, synthetic construct
SEFAYGSFMRSVTLPPGADE

SEQ ID NO: 134 - protein, 33 aa, synthetic construct
AEKAGGGGGAEKASEFAYGSFVRTVSLPVGADE

SEQ ID NO: 135 - protein, 47 aa, synthetic construct
AEKAGGGGGAEKASEFAYGSFVRTVSLPVGADEQYIKANSKFIGITE

SEQ ID NO: 136 - protein, 33 aa, synthetic construct
SEFAYGSFVRTVSLPVGADEAEKAGGGGGAEKA

SEQ ID NO: 137 - protein, 38 aa, synthetic construct
AEKAGGGGGSEFAYGSFVRTVSLPVGADEGGGGGAEKA

SEQ ID NO: 138 - protein, 52 aa, synthetic construct
AEKAGGGGGSEFAYGSFVRTVSLPVGADEGGGGGAEKAQYIKANSKFIGITE

SEQ ID NO: 139 - protein, 43 aa, synthetic construct
WRMYFSHRHAHLRSPGGGGGAEKAGGGGGQYIKANSKFIGITE

SEQ ID NO: 140 - protein, 52 aa, synthetic construct
WHWRHRIPLQLAGRAEKAGGGGGWRMYFSHRHAHLRSPQYIKANSKFIGITE

SEQ ID NO: 141 - protein, 21 aa, synthetic construct
YFPLQSYGFQPTNGVGYQPYR

SEQ ID NO: 142 - protein, 35 aa, synthetic construct
YFPLQSYGFQPTNGVGYQPYRQYIKANSKFIGITE

SEQ ID NO: 143 - protein, 34 aa, synthetic construct
YQAGSTPCNGVEGFNCYFPLQYIKANSKFIGITE

SEQ ID NO: 144 - protein, 21 aa, synthetic construct
YQAGSTPCNGVEGFNCYFPLQ

SEQ ID NO: 145 - protein, 19 aa, synthetic construct
NPDPNANPNVDPNANGGGG

SEQ ID NO: 146 - protein, 13 aa, synthetic construct
RKSIHLGPGRAFY

SEQ ID NO: 147 - protein, 13 aa, synthetic construct
KKGIAIGPGRTLY

SEQ ID NO: 148 - protein, 13 aa, synthetic construct
RKSIRIGPGQAFY

SEQ ID NO: 149 - protein, 13 aa, synthetic construct
RKRIRVGPGQTVY

SEQ ID NO: 150 - protein, 9 aa, synthetic construct
CATGIAVAG

SEQ ID NO: 151 - protein, 10 aa, synthetic construct
YYYYYGMDVW

SEQ ID NO: 152 - protein, 14 aa, synthetic construct
CATGYSSSWYFDYW

SEQ ID NO: 153 - protein, 15 aa, synthetic construct
CAKGYSYGYNWFDSW

SEQ ID NO: 154 - protein, 12 aa, synthetic construct [sequence omitted]

SEQ ID NO: 155 - protein, 23 aa, synthetic construct
CATGIAVAGQYIKANSKFIGITE

SEQ ID NO: 156 - protein, 24 aa, synthetic construct
YYYYYGMDVWQYIKANSKFIGITE

SEQ ID NO: 157 - protein, 28 aa, synthetic construct
CATGYSSSWYFDYWQYIKANSKFIGITE

SEQ ID NO: 158 - protein, 29 aa, synthetic construct
CAKGYSYGYNWFDSWQYIKANSKFIGITE

SEQ ID NO: 159 - protein, 26 aa, synthetic construct
CQQYNNWPPLTFQYIKANSKFIGITE

SEQ ID NO: 160 - protein, 36 aa, synthetic construct
ARDLICAQCATGYSSSWYFDYWQYIKANSKFIGITE

SEQ ID NO: 161 - protein, 53 aa, synthetic construct
WDYPKCDRATEVETPIRNEHYEECSCYCQQYNNWPPLTFQYIKANSKFIGITE

SEQ ID NO: 162 - protein, 58 aa, synthetic construct
WDYPKCDRATEVETPIRNEARDLICAQENQKLIANCATGIAVAGQYIKANSKDIGITE

SEQ ID NO: 163 - protein, 53 aa, synthetic construct
WDYPKCDRAENQKLIANARDLICAQCATGYSSSWYFDYWQYIKANSKFIGITE

SEQ ID NO: 164 - protein, 19 aa, synthetic construct
NPDPNANPNVDPNANGGGC

SEQ ID NO: 165 - protein, 24 aa, synthetic construct
NMMRFTSQPPNNNMMNYIMDPRTH

SEQ ID NO: 166 - protein, 12 aa, synthetic construct
  VARIANT 8: note = Any amino acid
STLNYMYXAHPF

SEQ ID NO: 167 - protein, 12 aa, synthetic construct
ISLSNIVDSQTP

SEQ ID NO: 168 - protein, 12 aa, synthetic construct
GFSVITGAAMFE

SEQ ID NO: 169 - protein, 46 aa, synthetic construct
NPDPNANPNVDPNANGGGCRKSIHLGPGRAFYQYIKANSKFIGITE

SEQ ID NO: 170 - protein, 50 aa, synthetic construct
  VARIANT 8: note = Any amino acid
STLNYMYXAHPFWRMYFSHRHAHLRSPGGGGGAEKAQYIKANSKFIGITE

SEQ ID NO: 171 - protein, 35 aa, synthetic construct
NMMRFTSQPPNNGGGGGAEKAQYIKANSKFIGITE

SEQ ID NO: 172 - protein, 58 aa, synthetic construct
NPDPNANPNVDPNANGGGCHSFKWLDSPRLRRKSIRIGPGQAFYQYIKANSKFIGITE

SEQ ID NO: 173 - protein, 55 aa, synthetic construct
AEKAGGGGGHSFKWLDSPRLRSEFAYGSFVRTVSLPVGADEQYIKANSKFIGITE

SEQ ID NO: 174 - protein, 7 aa, synthetic construct
QEINSSY

SEQ ID NO: 175 - protein, 7 aa, synthetic construct
APPHALS

SEQ ID NO: 176 - protein, 7 aa, synthetic construct
VVPTPPY

SEQ ID NO: 177 - protein, 7 aa, synthetic construct
SMPNPMV

SEQ ID NO: 178 - protein, 7 aa, synthetic construct
GLQQVLL

SEQ ID NO: 179 - protein, 7 aa, synthetic construct
ELAPDSP

SEQ ID NO: 180 - protein, 21 aa, synthetic construct
QEINSSYQYIKANSKFIGITE

SEQ ID NO: 181 - protein, 21 aa, synthetic construct
APPHALSQYIKANSKFIGITE

SEQ ID NO: 182 - protein, 21 aa, synthetic construct
VVPTPPYQYIKANSKFIGITE

SEQ ID NO: 183 - protein, 45 aa, synthetic construct
QEINSSYAEKAGGGGGWRMYFSHRHAHLRSPQYIKANSKFIGITE

SEQ ID NO: 184 - protein, 45 aa, synthetic construct
APPHALSAEKAGGGGGWRMYFSHRHAHLRSPQYIKANSKFIGITE

SEQ ID NO: 185 - protein, 45 aa, synthetic construct
VVPTPPYAEKAGGGGGWRMYFSHRHAHLRSPQYIKANSKFIGITE

SEQ ID NO: 186 - protein, 46 aa, synthetic construct
AEAKAGGGGGWRMYFSHRHAHLRSPQEINSSYQYIKANSKFIGITE

SEQ ID NO: 187 - protein, 45 aa, synthetic construct
WRMYFSHRHAHLRSPAPPHALSAEKAGGGGGQYIKANSKFIGITE

SEQ ID NO: 188 - protein, 44 aa, synthetic construct
QYIKANSKFIGITEWRMYFSHRHAHLRSAEKAGGGGGVVPTPPY

SEQ ID NO: 189 - protein, 57 aa, synthetic construct
QEINSSYAEKAGGGGGWRMYFSHRHAHLRSPGFSVITGAAMFEQYIKANSKFIGITE

SEQ ID NO: 190 - protein, 44 aa, synthetic construct
GNLFIAPAPPHALSWGVIHHPHYEECSCYQYIKANSKFLIGITE

SEQ ID NO: 191 - protein, 16 aa, synthetic construct
RQIKIWFQNRRMKWKK

SEQ ID NO: 192 - protein, 11 aa, synthetic construct
YGRKKRRQRRR

SEQ ID NO: 193 - protein, 20 aa, synthetic construct
HYRIKPTFRRLKWKYKGKFW

SEQ ID NO: 194 - protein, 46 aa, synthetic construct
GNLFIAPWGVIHHPHYEECSCYTEVETPIRNEQYIKANSKFIGITE

SEQ ID NO: 195 - protein, 55 aa, synthetic construct
WDYPKCDRATEVETPIRNEGNLFIAPWGVIHHPHYEECSCYQYIKANSKFIGITE

SEQ ID NO: 196 - protein, 62 aa, synthetic construct
SEFAYGSFVRTVSLPVGADEWRMYFSHRHAHLRSPAPPHALSAEKAGGQYIKANSKFIGITE

SEQ ID NO: 197 - protein, 66 aa, synthetic construct
SEFAYGSFVRTVSLPVGADETEVETPIRNEGNLFIAPWGVIHHPHYEECSCYQYIKANSKFIGITE

SEQ ID NO: 198 - protein, 53 aa, synthetic construct
GNLFIAPWGVIHHPHYEECSCYTEVETPIRNEQYIKANSKFIGITEAPPHALS

SEQ ID NO: 199 - protein, 53 aa, synthetic construct
APPHALSGNLFIAPWGVIHHPHYEECSCYTEVETPIRNEQYIKANSKFIGITE

SEQ ID NO: 200 - protein, 53 aa, synthetic construct
GNLFIAPWGVIHHPHYEECSCYAPPHALSTEVETPIRNEQYIKANSKFIGITE

SEQ ID NO: 201 - protein, 9 aa, synthetic construct
TYQRTRALV

SEQ ID NO: 202 - protein, 55 aa, synthetic construct
TYQRTRALVGNLFIAPWGVIHHPHYEECSCYTEVETPIRNEQYIKANSKFIGITE

SEQ ID NO: 203 - protein, 41 aa, synthetic construct
GNLFIAPWGVIHHPHYEECSCYTEVETPIRNETYQRTRALV

SEQ ID NO: 204 - protein, 55 aa, synthetic construct; identical to SEQ ID NO: 195